(19) United States
(12) Patent Application Publication
(10) Pub. No.: US 2023/0193192 A1
(43) Pub. Date: Jun. 22, 2023

(54) MEDIUM FOR THE GROWTH OF FASTIDIOUS BACTERIA

(71) Applicant: GEORGE MASON UNIVERSITY, Fairfax, VA (US)

(72) Inventors: Monique Louise van Hoek, Fairfax, VA (US); Stephen John Kassinger, Fairfax, VA (US)

(21) Appl. No.: 17/554,363

(22) Filed: Dec. 17, 2021

(51) Int. Cl. C12N 1/20

(52) U.S. Cl. CPC C12N 1/20; C12N 2500/24; C12N 2500/74

(57) ABSTRACT

The present invention relates to a composition comprising a growth medium comprising a carbon source, a nitrogen source, a sulphur containing amino acid and Ferric pyrophosphate; wherein the growth medium has no blood; wherein the growth medium is configured to allow growth of a living microorganism. Further, the invention also relates to method to prepare the composition.

### FIELD OF THE INVENTION

This invention relates to a composition comprising a medium for growth, isolation, enrichment, and recovery of fastidious microorganism. This invention particularly relates to growth of Francisella. This invention also relates to method to formulate the medium.

### BACKGROUND OF THE INVENTION

US20110177515A1 states, “Francisella is a genus of pathogenic, gram-negative bacteria. They are rod-shaped and non-motile. Francisella tularensis is the causative agent of tularemia. A disease endemic to the US. It has also been weaponized in the past. Tularemia is also known as “rabbit fever”, “deer-fly fever”, “Ohara fever” and “Francis disease.” The disease is endemic in North America, and parts of Europe and Asia. The most common modes of transmission are via arthropod vectors, waterborne infection, and by biting flies, particularly the deer fly Chrysops discalis. Other members of the genus Francisella include the species F. novicida and F. philomiragia.”

US20070066801A1 states, “Francisella tularensis, the agent causing tularemia, is a CDC category A agent and is considered as a major biological threat. F. tularensis has long been considered as a potential biological weapon. In the 1950's and the 1960's, the US military developed weapons that would disseminate F. tularensis aerosols and by the late 1960s, F. tularensis was one of several biological weapons stockpiled (2). A parallel effort by Soviet Union continued into the early 1990s and resulted in weapons production of F. tularensis strains engineered to be resistant to antibiotics and vaccines (3). F. tularensis is a good candidate for a biological weapon due to its very high infectivity. The infectious dose can be as low as 10 to 50 microorganisms if inhaled (4). In 1969, a World Health Organization expert committee estimated that an aerosol dispersal of 50 kg of virulent F. tularensis over a metropolitan area with 5 million inhabitants would result in 250,000 incapacitated casualties, including 19,000 deaths (5). Recently, the Centers for Disease Control and Prevention examined the expected economic impact of bioterrorist attacks and estimated that the total base cost to society of an F. tularensis aerosol attack would be $5.4 billion for every 100,000 persons exposed (6).

F. tularensis bacterium has several subspecies, with varying degrees of virulence. The tularensis subspecies (type A) is found predominantly in North America and is the most virulent of the known subspecies. Type A is associated with lethal pulmonary infections. The palearctica subspecies (also known as holarctica or type B) is found predominantly in Europe and Asia, and rarely leads to fatal disease. A third subspecies, novicida, has been characterized as a relatively nonvirulent strain.

Severity of disease can vary with subspecies of F. tularensis, discrimination among subspecies is a critical concern. Thus, there is a need in the art for assays and other aspects related to the rapid detection of Francisella and characterization of the Francisella species and subspecies.

These fastidious bacteria having severe requirements for growth environment and nutrition. They are usually difficult to grow in the ordinary environment, and even if they can grow, they are difficult to grow typical colonies which makes them difficult to characterize. Thus, fastidiousness is often practically defined as being difficult to culture, by any method yet tried, given that they are obligate parasites of hosts. Current studies rely on laborious and time-consuming culture medium.

US20070066801A1 discloses “Rapid diagnostic testing for tularemia is not widely available (reviewed in 41, 42). The organism can be isolated from blood, sputum, skin, or mucosal membrane lesions of an affected individual, but microbiologic diagnosis can be difficult due to unusual growth requirements for Francisella strains and/or due to the overgrowth of commensal bacteria.”

Historically, Francisella media yielded poor bacterial growth in vitro (57). In order to successfully grow large amounts of robust Francisella species animal hosts were used (58). This in part drove the creation of Francisella selective media. Some of the earliest media used to isolate Francisella species selectively were based around Thayer-Martin like agars designed to isolate Neisseria species (58). Thayer-Martin agars have a Mueller-Hinton base with added chocolatized sheep blood (59). One of the first Thayer-Martin like media that was designed to isolate Francisella had colistin, trimethoprim, nystatin and lincomycin added to it (58). This medium was shown to be able to isolate Francisella from select oral microbiota inconsistently. Another Francisella selective medium was designed with the intent of isolating Francisella from deceased animals (29). This medium consisted of a cysteine heart agar base, sheep's blood, colistin, amphotericin, lincomycin, trimethoprim, and ampicillin (29). When this medium was shown to not be suitable for environmental isolation, the same group developed a medium with the same base with added polymyxin B, amphotericin B, cefepime, cycloheximide and vancomycin. Another group which was studying recovery of seeded Francisella from environmental water samples has successfully used cysteine heart agar with rabbits blood and added polymyxin B and penicillin (60).

The existing growth medium is generally prepared by mixing sterile sheep blood or rabbit blood or horse blood with a sterile agar culture medium according to a proportion, and then putting the mixture into a disposable sterile plastic plate to prepare a disposable plate for culturing (growing and propagating) bacteria.

Problems with currently available media include only being available in solid form, being extremely dark, expensive, difficult to prepare, time intensive to prepare, requiring refrigeration, and resulting in extremely slow growth.

Due to this, there is a long-felt need, there is need of medium cost effective, no refrigeration needed, easily helps to grow in a less time. To solve the long-standing problem, the present invention leads to a composition comprising a growth medium for the growth of fastidious organism but not limited to Francisella.

### SUMMARY

Cultivation of the most fastidious Francisella strains in monoculture is generally problematic including but not limited only being available in solid form, being extremely dark, being expensive, being difficult to make, being time intensive to make, requiring refrigeration, and resulting in extremely slow growth.

Due to the extremely infectious nature of Francisella tularensis, it is studied at biosafety level BSL-3, and is classified as a Category A, Tier 1 threat agent. The Francisella tularensis tularensis type A biotype “type strain” is an uncommon strain NIH B38. This strain has lost virulence through laboratory passage and can be used in a BSL-2 laboratory. While the NIH B38 strain is genetically more similar to the fully virulent Francisella tularensis tularensis SchuS4 than other model organisms such as Francisella novicida U112 or Francisella holarctica LVS, NIH B38 is seldom utilized as a model since it does not grow in standard liquid media used for tularemia research.

Here we bridge the gap between BSL-2 and BSL-3 Francisella studies by developing a novel liquid medium capable of growing NIH B38 as well as SchuS4, named FIRE (“Francisella Isolation Recovery and Enrichment”) medium. We demonstrate that FIRE medium was able to grow the fastidious NIH B38, and it was also able to grow F. holarctiva LVS and F. novicida faster than more common media such as TSB-C and BHI.

During the creation of FIRE medium, we noticed that alternate methods of sterilization differentially influenced the growth of various Francisella strains. When autoclaved, our medium would support the growth of F. novicida U112 and F. holarctica LVS, but neither F. tularensis NIH B38 nor SchuS4 would grow. Using elimination and addition experiments coupled with quadrupole time of flight mass spectrometry, we discovered that an inhibitory growth product was created while autoclaving which was selectively preventing the growth of and NIH B38 and likely SchuS4. This highlights the advantage to using the NIH B38 strain, as this inhibitor would not have been discovered using either F. novicida U112 or F. holarctica LVS.

To demonstrate the usefulness of our new FIRE medium, we used it develop a selective medium for Francisella isolation. MIC and Disk Diffusion based antibiotic resistance testing determined that all the strains of Francisella tested had equal or greater resistance to colistin (polymyxin E) than to polymyxin B.

Using FIRE selective medium (with colistin), we were able to recover NIH B38 from complex mixtures of microbes. We also used this selective medium to investigate percentage recovery of Francisella relative to other Francisella selective media. We discovered that both the strain of Francisella and its previous growth conditions influenced which selective medium was able to recover the most Francisella.

In summary, we have developed a new growth medium, FIRE, for the growth of Francisella tularensis. This media does not use blood products, is easy to make, stores well, and is significantly less expensive than the current options. We have successfully enabled the use of the BSL-2 type strain F. tularensis NIH B38 for tularemia studies and further demonstrated the benefit of using NIH B38 by discovering a novel inhibitory compound that inhibits the growth of F. tularensis tularensis SchuS4.

The present invention relates to a growth medium that is cost effective, no refrigeration needed, easily helps to grow microorganism in a less time.

The present invention does not require mixing of sterile sheep blood or rabbit blood or horse blood with a sterile agar culture medium according to a proportion.

An embodiment relates to a composition comprising a growth medium comprising a carbon source, a nitrogen source, a sulfur containing amino acid and a Ferric salt; wherein the growth medium has no blood; wherein the growth medium is configured to allow growth of a living microorganism.

In an embodiment, the ferric salt is ferric pyrophosphate.

In an embodiment, the sulfur containing amino acid comprises cysteine.

In an embodiment, the growth medium is configured to growth of Francisella.

In an embodiment, the carbon source comprises at least one of Lactose, Sucrose, Dextrose.

In an embodiment, the nitrogen source comprises at least one of Peptone, Beef extract, Casein Hydrolysate, Yeast Extract.

In an embodiment, the growth medium further comprising a buffer.

In an embodiment, the buffer comprises at least one of HEPES, PIPES, MOPS, a Dibasic Phosphate solution, a monophasic Phosphate solution.

In an embodiment, the growth medium further comprises a salt to regulate an osmotic stress.

In an embodiment, the salt comprises sodium chloride.

In an embodiment, the growth medium is configured to allow growth of a BSL-2 strain of Francisella and a BSL-3 strain of Francisella.

In an embodiment, the growth medium further comprises thiamine.

In an embodiment, the growth medium further comprises spermine.

In an embodiment, the growth medium is configured to convert into a selective growth media.

In an embodiment, the selective growth medium comprises an antibiotic.

In an embodiment, the selective growth medium is configured to isolate Francisella from a mixture of the living microorganism.

In an embodiment, the growth medium configured to enrich Francisella from a mixture of the living microorganism.

In an embodiment, the growth medium is configured to recover Francisella from a mixture of the living microorganism.

In an embodiment, the antibiotic comprises a cyclic peptide antibiotic.

In an embodiment, the cyclic peptide antibiotic comprises polymyxin and/or colistin.

In an embodiment, the antibiotic comprises at least one of polymyxin, colistin, Amphotericin, Cefepime, Cycloheximide, Vancomycin.

In an embodiment, the nitrogen source is about 40 g/l to about 60 g/l.

In an embodiment, the carbon source is about 5 g/l to about 30 g/l.

In an embodiment, the Ferric pyrophosphate is more than zero and less than 1 g/l.

In an embodiment, the growth medium has pH about 5 to about 7.

In an embodiment, the growth medium does not require refrigeration for storage.

An embodiment relates to a method comprises: mixing a nitrogen source, cysteine, a salt to regulate to osmotic water and a buffer to form a mixture; adding water to mixture and autoclaving a dissolved mixture in the water; and adding ferric pyrophosphate and a carbon source to form a growth medium; wherein the growth medium has no blood and is configured to support growth of Francisella.

An embodiment relates to the method wherein the growth media is capable to be converted into a selective growth media by addition of an antibiotic.

An embodiment relates to the method wherein the antibiotic comprises at least one of cyclic peptide antibiotic comprises polymyxin and/or colistin, Amphotericin, Cefepime, Cycloheximide, Vancomycin.

An embodiment relates to the method wherein the carbon source comprises at least one of Lactose, Sucrose, Dextrose.

An embodiment relates to the method wherein the nitrogen source comprises at least one of Peptone, Beef extract, Casein Hydrolysate, Yeast Extract.

An embodiment relates to the method wherein the growth medium is configured to allow growth of a BSL-2 strain of Francisella and a BSL-3 strain of Francisella.

An embodiment relates to the method wherein the mixture further comprises thiamine.

An embodiment relates to the method wherein the mixture further comprises spermine.

An embodiment relates to the method wherein the growth medium has a pH of about 5 to about 7.

In an embodiment, the invention relates to development of a new growth medium, FIRE (Francisella isolation, enrichment and recovery medium), for the growth of Francisella tularensis. In an embodiment, this medium does not use blood products, is easy to make, stores well, and is significantly less expensive than the current options. A further embodiment relates to the use of the BSL-2 type strain F. tularensis NIH B38 for tularemia studies and further demonstrate the benefit of using NIH B38 by using it to discover of a novel compound that inhibits the growth F. tularensis tularensis SchuS4.

In an embodiment, FIRE enables the growth of Francisella.

Yet another embodiment relates to the growth of fastidious genera but not limited to Mycobacteria, Yersinia, Coxiella, Rickettsia, Salmonella, Brucella, Listeria, Shigella, Chlaymdia, Shigella, Legionella, Helicobacter, Neisseria.

In an embodiment, FIRE may support robust growth in all the genera of fastidious microorganisms.

In another embodiment, the medium may support the growth of fastidious, facultative intracellular, and obligate intracellular bacteria.

In an embodiment, FIRE supports better growth of fastidious microorganisms as compared to but not limited to Chamberlain's chemical defined media, BMFC, BVFH, BHIC, Chocolate agar plates.

In an embodiment, ferric salt comprises ferric pyrophosphate.

In an embodiment, media comprises Iron salt in tri valency state of iron.

In an embodiment, media comprise iron salt in divalency state of iron.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The figures are furnished with the application to understand the invention sought to be patented. It shall not be construed as only way to perform the invention has sought to be patented.

FIG. 1A: shows growth rate of Francisella novicida in various broth media as measured at OD 600 nm, as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the top line (FIRE, Red), the cysteine (yellow) and BMFC (brown) were overlapping and are in the middle of the graph, while BHI (blue) showed the lowest rate of growth (bottom line).

FIG. 1B: shows growth rate of E holarctica LVS in various broth media as measured by OD600 nm, as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the top line (FIRE, Red), the cysteine (yellow) and BMFC (brown) were overlapping and are in the middle of the graph, while BHI (blue) showed the lowest rate of growth (bottom line).

FIG. 1C: shows growth rate of F. tularensis tularensis NIH B38 in various broth media as measured by OD600 nm, as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the top line (FIRE, Red), and BMFC (brown) is the next line below. Cysteine (Yellow) and BHI (blue) showed no growth (bottom lines along X axis).

FIG. 1D: shows the growth of Francisella novicida in various media as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the BMFC (brown), which reached the highest peak, and then fell to the lowest line as time proceeded. The BHI (blue) and TSBC (yellow) had the second highest peaks and were overlapping in the middle of the graph. The FIRE medium (Red) had the lowest early peak of growth, and continues to grow over time, peaking much later than the other media. One the peak was achieved in this media, a slow but steady decrease of OD600 was observed.

FIG. 1E: shows the growth of F. holarctica LVS in various media as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the cysteine (yellow) and BHI media (blue) were overlapping and are in the top of the graph at 12 hours, while BMFC (brown) showed the lowest rate of growth and a slow peak of growth at 36 hours (bottom line). FIRE medium (Red) has a long growth curve and slow peak, with maximal peak at 48 hours and then a slow decline of OD600 after that point.

FIG. 1F: shows the growth of F. tularensis tularensis NIH B38 strain in various media as measured by absorbance over time in tryptic soy broth with 0.1% (w/v) cysteine (Yellow), BMFC (Brown), FIRE (Red), and brain heart infusion (Blue). Standard deviations are displayed in light grey. The highest growth rate as measured by absorbance (OD600) was the FIRE media (Red) which achieved the highest level of growth. BMFC media (brown) showed the next highest level of growth and a slow peak of growth at 36 hours (bottom line). Cysteine (Yellow) and BHI (blue) media showed no growth (bottom lines along X axis).

FIG. 2: shows nutrient limitation testing. F. tularensis tularensis NIH B38 growth was compared in filter-sterilized FIRE (FS), autoclaved FIRE (AF), along with 2× filter-sterilized FIRE diluted 1:2 into autoclaved FIRE (AF/FF) and autoclaved FIRE diluted 1:2 with deionized water (AF/H₂O)

FIG. 3A: shows single and double elimination experiments. NIH B38 growth was compared in filter-sterilized FIRE (FS), autoclaved FIRE (AF), along with autoclaved FIRE missing each of its seven ingredients Dextrose (ΔDex), Meat peptone (ΔMP), Sodium Chloride (ΔNaCl), Sodium Phosphate monobasic monohydrate (ΔNaPO4 MM), Sodium Phosphate dibasic anhydrous (ΔNaPO4 DA), Cysteine (ΔCys), ferric pyrophosphate (ΔFe) and finally autoclaved FIRE missing both dextrose and iron pyrophosphate (ΔDex ΔFe) as well as autoclaved FIRE missing dextrose and iron pyrophosphate with filter sterilized dextrose being added in after autoclaving (ΔDex ΔFe+Dex).

FIG. 3B: shows single and double elimination experiments. SchuS4 (B) growth was compared in filter-sterilized FIRE (FS), autoclaved FIRE (AF), along with autoclaved FIRE missing each of its seven ingredients Dextrose (ΔDex), Meat peptone (ΔMP), Sodium Chloride (ΔNaCl), Sodium Phosphate monobasic monohydrate (ΔNaPO4 MM), Sodium Phosphate dibasic anhydrous (ΔNaPO4 DA), Cysteine (ΔCys), ferric pyrophosphate (ΔFe) and finally autoclaved FIRE missing both dextrose and iron pyrophosphate (ΔDex ΔFe) as well as autoclaved FIRE missing dextrose and iron pyrophosphate with filter sterilized dextrose being added in after autoclaving (ΔDex ΔFe+Dex).

FIG. 4: shows determination of reactants in inhibitor formation. The growth of NIH B38 was compared in filter-sterilized FIRE (FS), autoclaved FIRE (AF), along with autoclaved FIRE missing Dextrose and Ferric Pyrophosphate (AF ΔΔ), autoclaved FIRE missing dextrose and ferric pyrophosphate with added back in autoclaved dextrose (AF ΔΔ [Dex]), autoclaved FIRE missing dextrose and ferric pyrophosphate with added back in autoclaved ferric pyrophosphate (AF ΔΔ [Fe]), autoclaved FIRE missing dextrose and ferric pyrophosphate with added back in autoclaved ferric pyrophosphate as well as added back in autoclaved dextrose (AF ΔΔ [Dex][Fe]), each was which was autoclaved alone, or finally autoclaved FIRE missing dextrose and ferric pyrophosphate with added back in autoclaved ferric pyrophosphate and dextrose which were autoclaved together (A ΔΔ [DexFe]).

FIG. 5: Generation of spontaneous resistance to colistin and polymyxin B. The inhibition of Francisella lawn formation/appearance of resistance colonies within a zone of resistance when exposed to colistin or polymyxin B. The black arrow indicates resistant colonies.

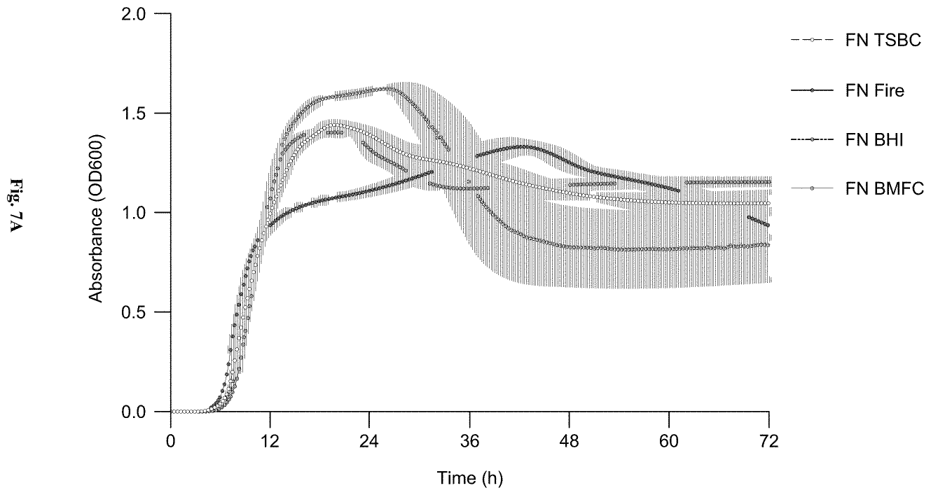

FIG. 6A: Colistin Toxicity in a Waxworm Model. Colistin was dissolved into cell culture PBS. Various doses of colistin were injected into waxworms. Survival of waxworms was monitored daily.

FIG. 6B: Polymyxin B Toxicity in a Waxworm Model. Polymyxin B was dissolved into cell culture PBS. Various doses of polymyxin B were injected into waxworms. Survival of waxworms was monitored daily.

FIG. 7A: Growth Rate of Francisella novicida in Various Media. Here is the growth of Francisella novicida as measured by absorbance over time in tryptic soy broth with 0.1% (w/v) cysteine (Yellow), BMFC (Brown), FIRE (Red), and brain heart infusion (Blue). Standard deviations are displayed in light grey.

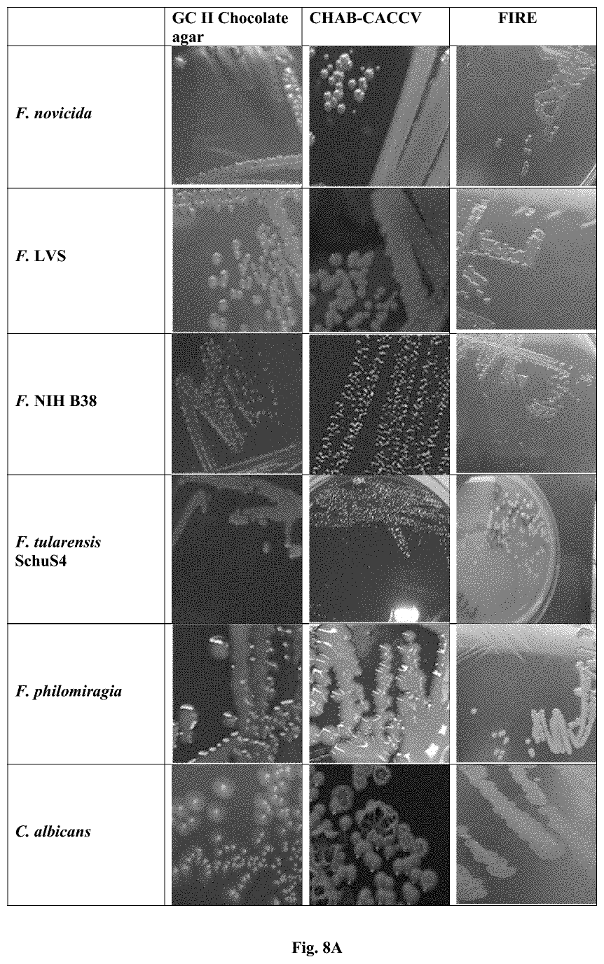

FIG. 7B: Growth Rate of Francisella LVS in Various Media. Here is the growth of Francisella LVS as measured by absorbance over time in tryptic soy broth with 0.1% (w/v) cysteine (Yellow), BMFC (Brown), FIRE (Red), and brain heart infusion (Blue). Standard deviations are displayed in light grey.

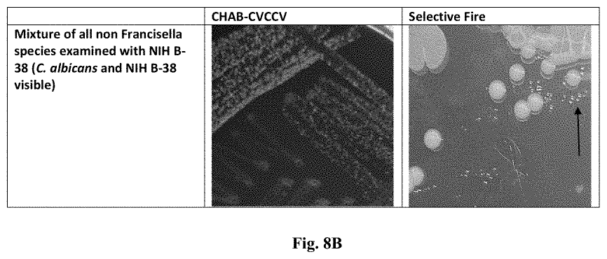

FIG. 7C: Growth Rate of Francisella NIH B38 in Various Media. Here is the growth of Francisella NIH B38 as measured by absorbance over time in tryptic soy broth with 0.1% (w/v) cysteine (Yellow), BMFC (Brown), FIRE (Red), and brain heart infusion (Blue). Standard deviations are displayed in light grey.

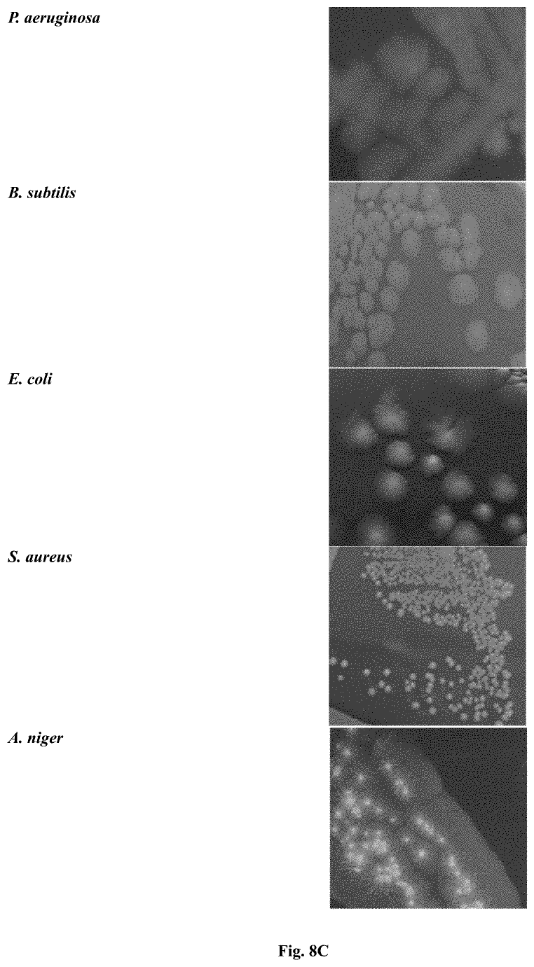

FIG. 8A: shows colony morphology of samples able to grow of Francisella selective media. The appearance of all Francisella species tested and C. albicans on both permissive GC II Chocolate agar as well as selective CHAB-CACCV and FIRE.

FIG. 8B: shows culturing of complex biological samples on Francisella selective media. Pictured are the results of all non-Francisella species examined, mixed, and pleated with NIH B38. The CHAB-CVCCV plate shows a mixture of C. albicans (White) mixed with NIH B38 (Magenta). The FIRE plate shows the smaller NIH B38 (Indicated by Black Arrow) with the larger C. albicans colonies

FIG. 8C: shows selective recovery-input viability confirmations. Pictured are the inputs for the tested inputs for the selective recovery test that did not yield colonies on the selective medium. Each of the species grew successfully on GC II chocolate agar within 2 days.

FIG. 9: shows relative culturing/recovery rates of various Francisella media. The recovery of several media of Francisella CFU/ml of F. novicida (A), LVS (B), NIH B38 (C) or SchuS4 (D) from either liquid media (1) or GC II Chocolate lawns (2) when plated on GC II Chocolate agar, CHAB-PACCV, CHAB-CACCV and Selective FIRE.

### DETAILED DESCRIPTION

### Definitions and General Techniques

For simplicity and clarity of illustration, the drawing illustrates the general manner of construction. Descriptions and details of well-known features and techniques may be omitted to avoid unnecessarily obscuring the present disclosure. Additionally, elements in the drawing are not necessarily drawn to scale. For example, the dimensions of some of the elements in the figures may be exaggerated, relative to other elements, to help improve the understanding of embodiments of the present disclosure. The same reference numeral in different figures denotes the same element.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As defined herein, “about” can, in some embodiments, mean within plus or minus five units of the stated value. In other embodiments, “about” can mean within plus or minus three units of the stated value. In further embodiments, “about” can mean within plus or minus two units of the stated value. In yet other embodiments, “about” can mean within plus or minus one unit of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment”, or “in an embodiment”, or the like, in places throughout the specification are not necessarily all referring to the same embodiment.

Furthermore, the features, structures, or characteristics may be combined in any suitable manner in one or more embodiments. Thus, the features, structures, or characteristics illustrated or described in connection with one embodiment may be combined, in whole or in part, with the features structures, or characteristics of one or more other embodiments without limitation. Such modifications and variations are intended to be included within the scope of the present invention.

The following terms and phrases, unless otherwise indicated, shall be understood to have the following meanings.

Biosafety level: A biosafety level (BSL), or pathogen/protection level, defined herein is a set of biocontainment precautions required to isolate dangerous biological agents in an enclosed laboratory facility. The levels of containment range from the lowest biosafety level 1 (BSL-1) to the highest at level 4 (BSL-4). In the United States, the Centers for Disease Control and Prevention (CDC) have specified these levels. In the European Union, the same biosafety levels are defined in a directive. In Canada the four levels are known as Containment Levels.

BSL-2: Biosafety level 2 or BSL-2 described herein relates to facilities applicable to clinical, diagnostic, teaching and other laboratories in which the broad spectrum of indigenous agents of moderate risk is worked that are present in the community and associated with human disease of varying severity. With good Microbiology techniques, these agents can be used safely in activities performed on an open work surface, as long as the potential to produce splashes or aerosols is low. Hepatitis B Virus, HIV, Salmonella and the Toxoplasma species are representative of the microorganisms assigned to this level of containment. The level of biosafety 2 is appropriate when working with any blood, body fluids, tissues or fundamentally human cell lines derived from humans in which the presence of an infectious agent may be unknown. (Laboratory personnel working with materials derived from humans should consult the OSHA bloodborne pathogen standard for specific necessary precautions.) The main hazards for personnel working with these agents refer to accidental percutaneous or mucous membrane exposures, or ingestion of infectious materials. Extreme precautionary measures should be taken with contaminated needles or sharp instruments. Even though it is known that organisms normally handled at a biosafety level 2 are not transmissible by aerosols, procedures with aerosols or with a high possibility of producing splashes that may increase the risk of such exposure to personnel should be carried out in containment equipment primary or in devices such as a BSC or safety centrifuge cups. Other primary barriers should be used as appropriate, such as splash shields, face protection, gowns and gloves. There must be secondary barriers such as hand wash basins and waste decontamination facilities to reduce possible environmental contamination.

BSL-3: Biosafety level 3 or BSL-3 described herein relates to facilities applicable to clinical, diagnostic, teaching, research or production facilities where indigenous and exotic agents are worked with a potential for respiratory transmission and that can cause a serious and potentially lethal infection. Mycobacterium tuberculosis, St. Louis Encephalitis Virus and Coxiella burnetii are representative of the microorganisms assigned to this level. The main hazards for personnel working with these agents concern self-inoculation, ingestion and exposure to infectious aerosols. At a biosafety level 3, greater emphasis is placed on primary and secondary barriers to protect personnel from contiguous areas, the community and the environment from exposure to potentially infectious aerosols. For example, all laboratory manipulations should be done in a BSC or other closed equipment, such as a gas-tight aerosol generation chamber. Secondary barriers to this level include controlled access to the laboratory and ventilation needs to minimize the release of infectious aerosols from the laboratory.

Strain: A strain described herein, is a genetic variant, a subtype or a culture within a biological species. Strains are often seen as inherently artificial concepts, characterized by a specific intent for genetic isolation. This is most easily observed in microbiology where strains are derived from a single cell colony and are typically quarantined by the physical constraints of a Petri dish. Strains are also commonly referred to within virology, botany, and with rodents used in experimental studies.

Category A: The U.S. Centers for Disease Control and Prevention (CDC) breaks biological agents into three categories: Category A, Category B, and Category C. Category A agents pose the greatest threat to the U.S. Criteria for being a Category “A” agent include high rates of morbidity and mortality; ease of dissemination and communicability; ability to cause a public panic; and special action required by public health officials to respond. These infectious agents would produce the worst casualties if they were to be released in a biological attack on a population. Category A agents include anthrax, botulism, plague, smallpox, viral hemorrhagic fevers, tularemia.

Biotype: The term biotype described herein, is used to designate populations that do not have morphological distinctions but have other useful attributes for separating them from other populations. Genetics and Molecular Biology but not limited to, using the RAPD markers are generally used to differentiate genetic variations and characterization between the new biotypes of the species.

Type A of Francisella: There are a variety of subspecies and biotypes of F. tularensis, but they all have greater than 95% DNA sequence identity. Although the type A and type B biotype strains are highly infectious, only type A strains, which are found exclusively in North America, cause significant mortality in infected humans. Most genes in the FPI (pathogenicity island of E tularensis) encode proteins with amino acid sequences are highly conserved between high- and low-virulence strains. One of the FPI genes is present in highly virulent type A of F. tularensis, absent in moderately virulent type B. In an embodiment, the “type strain” deposited for the fully virulent (type A) Francisella tularensis tularensis at BEI resources is an avirulent less-commonly used strain of Francisella known as Francisella tularensis subsp. tularensis, strain NIH B38 (NR50, ATCC6223, FSC230 BEIResources.org)

Tier 1 threat agent: In the interest of public health and safety, especially where a sample may contain target agent(s) that are thought to be a threat to the health of humans, animals or plants, causing societal disruption and economic harm are known as threat agent. According to CDC, Tier 1 threat agents and toxins require additional security measures to be implemented including the addition of pre-access suitability assessments, extra access controls, and extra barriers. These extra measures are intended to safeguard Tier 1 threat agents and toxins further from theft, loss, or release. The list of Tier 1 select agents and toxins includes: Bacillus anthracis, Bacillus cereus Biovar anthracis, Botulinum neurotoxins, Botulinum neurotoxin producing species of Clostridium, Burkholderia mallei, Burkholderia pseudomallei, Ebola virus, Foot-and-mouth disease virus, Francisella tularensis, Marburg virus, Rinderpest virus, Variola major virus (Smallpox virus), Variola minor virus (Alastrim), Yersinia pestis.

Resistant colonies: Resistant colonies are the colonies of the microorganisms which are resistant to a particular thing (e.g., drug, stress or gas) at a particular concentration, which could be either due to the transposon, or due to spontaneous mutations. In an embodiment, resistant colonies are the microbial colonies resistant to the cyclic peptide antibiotics.

Medium/Media: An environment containing or suitable for supporting microorganisms, including, but not limited to, broths, agar, cultures, foods, beverages, cell suspensions, biological tissue, biological fluids, inorganic surfaces, organic surfaces, substrates, living cells, host cells, diagnostic assays, and other solid, liquid, matrix, gelatinous, or gaseous environments.

Growth: In an embodiment, the term “Growth” refers to the increase in no. of cells or increase in size of a cell of the microorganism. Growth often leads to the increase in the turbidity of the liquid media or the appearance of the microbial colonies on the solid media.

Growth medium: The term “growth medium” as used herein, should be taken broadly to mean any medium which is suitable to support growth of a microbe. By way of an example, the media may be natural or artificial including, but not limited to, broth or solidified solutions. It should be appreciated that the media may be used alone or in combination with one or more other media. It may also be used with or without the addition of exogenous nutrients, antibiotics, vitamins, minerals and physical support systems for microbial growth.

Enrichment media: The term enrichment generally refers to increasing the ratio of the number of cells of a target/specific cell subpopulation present to the total number of cells present in a mixture of cells. “Enrichment medium” herein generally refers to a solution or broth or solidified medium in which to bathe and incubate cells that can be used to supply the cells with nutrients, such that enrichment of a target cell subpopulation can be achieved. Enrichment media can contain specific ligand-based signaling cues that can potentiate cell division of targeted cell. The exact composition of the enrichment medium can vary based on the cell type to be captured and enriched.

Isolation media: The terms “isolate”, “isolated”, “isolation” and like terms should be taken broadly. These terms are intended to signify separation of a microorganism(s) at least partially or completely from at least one of the materials with which it is associated in a particular environment (for example soil, water, plant tissue). “Isolate”, “isolated” and like terms should not be taken to indicate the extent to which the microorganism(s) has been purified. Media which is used with the intention to “isolate”, “isolated”, “isolation” is Isolation media. It is as used herein, “isolate,” “isolated,” “isolated microbe,” and like terms, are intended to mean that the one or more microorganisms has been separated from at least one of the materials or a mixture of living microorganism with which it is associated in a particular environment (for example soil, water, animal tissue). Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an acceptable carrier. For example: Media with a high salt concentration will select for halophiles. In an embodiment, cyclic peptide antibiotics are used isolate the resistant species of Francisella.

Recovery media: The terms “recovery”, “recovered” and like terms should be taken broadly. These terms are intended to mean that the one or more microorganism(s) has been recovered from their associated environmental condition. Microbe could be recovered from condition such as but not limiting to, after a particular condition i.e., after genome editing, molecular procedure, under physical or chemical stress or from lyophilized cells or glycerol stock etc. The medium which helps in recovery of microbes is called as a recovery media. Electroporation or transfection takes place in the transformation module, then the cells are transferred to a recovery media that optionally includes selection of the cells containing the one or more genomic edits. After recovery/editing/selection, the cells may be retrieved and used directly for research or stored for further research, or another round (or multiple rounds) of genomic editing can be performed by repeating the editing steps within the instrument and recovery media helps in all these procedures.

Endemic: Endemic relates to a disease that majorly exists in a certain place, in a hidden or shown way.

Monoculture: As the term described herein, an essentially pure culture containing only microbial cells of a single species, variety, or type descended from the originally lodged microbial cell but no other cells.

FIRE: FIRE is an abbreviation for Francisella Isolation Recovery and Enrichment (FIRE) media. This term should be considered broadly. This media could be used for either isolation, recovery, enrichment, or combination of these activity for microbes such as but not limited to Francisella. In an embodiment, FIRE medium is according to any embodiments of this invention.

CAMHB: CAMHB is an abbreviation for Cation adjusted Mueller-Hinton broth.

BVFH: Brain heart infusion broth supplemented with 2% Vitox, 10% Fildes and 1% histidine (BVFH).

BHIC: Brain heart infusion media with cysteine.

Tularemia: Tularemia, also known as rabbit fever, is an infectious disease caused by the bacterium Francisella tularensis. Symptoms may include fever, skin ulcers, and enlarged lymph nodes. Occasionally, a form that results in pneumonia or a throat infection may occur. The bacterium is typically spread by ticks, deer flies, or contact with infected animals. It may also be spread by drinking contaminated water or breathing in contaminated dust. Diagnosis is by blood tests or cultures of the infected site

Biodefense: Biodefense generally refers to measures to restore biosecurity to a group of organisms who are, or may be, subject to biological threats or infectious diseases. Biodefense is frequently discussed in the context of biowar or bioterrorism and is generally considered a military or emergency response term. Biodefense uses medical measures to protect people against bioterrorism. This includes medicines and vaccinations. It also includes medical research and preparations to defend against bioterrorist attacks.

Microorganism: The terms “microorganism” and “microbe” should be taken broadly and are used interchangeably herein and refer to any microorganism that is of the domain Bacteria, Eukarya or Archaea. Microorganism types include without limitation, bacteria (e.g., Mycoplasma, coccus, Bacillus, Rickettsia, spirillum), fungi (e.g., filamentous fungi, yeast), nematodes, protozoans, archaea, algae, dinoflagellates, protists, viruses (e.g., bacteriophages), viroids and/or a combination thereof. Organism strains are subtaxons of organism types, and can be for example, a species, sub-species, subtype, genetic variant, pathovar or serovar of a particular microorganism.

Facultative: Terms such as “Facultative” and “facultatively” should be considered broadly. It signifies microbes that are capable to thrive in more than one condition. For example, a facultative anaerobes or microbes as opposed to strict or obligatory anaerobes, generally prefer an oxygen environment but are capable of living and growing in its absence.

Intracellular: The term relates to existing, occurring, or functioning within a cell. The cells include mammalian cells, such as but are limited to rabbits, rodents and human cells. In an embodiment, Francisella is a facultative intracellular pathogen of phagocytic eukaryotic cells.

Culture: The term herein described as growing a population of microbial cells under suitable conditions for growth, in a liquid or solid medium. In an embodiment, culture here means ‘microbial culture’. A microbiological culture, or microbial culture, is a method of multiplying microbial organisms by letting them reproduce in predetermined medium under controlled laboratory conditions.

Fastidious: A fastidious organism is any organism that has complex or particular nutritional requirements. In other words, a fastidious organism will only grow when specific nutrients are included in its medium. The more restrictive term fastidious microorganism is used in microbiology to describe microorganisms that will grow only if special nutrients are present in their culture medium. Thus, fastidiousness is often practically defined as being difficult to culture, by any method yet tried. Some fastidious microbial species' requirements for life include not only particular nutrients but chemical signals of various kinds, some of which depend, both directly and indirectly, on other species being nearby. Thus, not only nutrient requirements but other chemical requirements can stand in the way of culturing species in isolation. In an embodiment, fastidious here relates to the fastidious microbes. In an embodiment, fastidious microbes are obligate parasite. One bottleneck (e.g., a major bottleneck) in characterizing the fastidious microbes is their inability to grow outside their natural hosts, as they are obligate parasites. It is estimated that >99% of microorganisms from any environment are non-cultivable in the laboratory. Numerous attempts have been made to create suitable artificial growth media and culture conditions for cultivating fastidious microbes.

Broth: The terms ‘Broth’ and ‘Broth media’ are used herein interchangeably. Broth is generally defined as a liquid medium containing nutrients. These nutrients could be used for culture and growth of microbes.

Solidified media: As described herein, broth is solidified using a solidified agent but not limited to agar, Gel-gro, polyol gel, carrageenan, calcium alginate, Pluronic polyol F127, gelrite etc.

Solidifying agent: The term herein used for the agents used for solidifying the media. Different solidifying agents are used generally on the basis of the temperature at which the media has to be kept. Few examples of solidifying agents with their solidifying temperature, without limitation are: Gelatin sets at about 25° C.; agar, the most commonly used solidifying agent for culture media, sets at about 45° C. and Gelrite has a holding temperature of 60°. Silica gel is tedious to prepare. Among other gelling agents which have been used for specialized applications are methyl cellulose, Gel-gro, Polycell cellulose paste, alginate gel. Semi-solid gels and precipitates such as AlPO₄ are also used for the isolation of bacteria.

Agar: Agar herein defined for the extract from algae are known as agarophytes, where it forms the supporting structure in the cell walls of certain species and is released on boiling. Agarophytes generally belong to Rhodophyta (red algae) phylum. It is a mixture of two components: the linear polysaccharide agarose, and a heterogeneous mixture of smaller molecules called agaropectin.

Solution: It is a mixture of two or more substances in relative amounts that can be varied continuously up to what is called the limit of solubility. The term solution is commonly applied to the liquid state of matter, but solutions of gases and solids are possible.

2× solution: A solution having double concentration relative to the solution which is generally prepared from those substances. In an embodiment, 2× solution of dextrose is prepared.

Carbon source: A carbon source has a dual role in biosynthesis and energy generation, with carbohydrates being the usual carbon source for microbial fermentation processes. In an embodiment, carbon source can be inorganic, organic, solid, liquid or any gaseous source. In an embodiment, carbon sources are: (1) monosaccharides—glucose, dextrose, fructose and galactose; (2) disaccharides—maltose, sucrose, lactose and trehalose; and (3) polysaccharides.

Lactose: Lactose is a disaccharide. It is a sugar composed of galactose and glucose subunits and has the molecular formula C₁₂H₂₂O₁₁.

Sucrose: Sucrose is a common sugar. It is a disaccharide, a molecule composed of two monosaccharides: glucose and fructose. Sucrose is produced naturally in plants, from which table sugar is refined. It has the molecular formula C₁₂H₂₂O₁₁.

Dextrose: Dextrose is the name of a simple sugar. It can be made from different food such as corn. Dextrose is also referred as corn sugar. Dextrose is chemically identical to glucose, or blood sugar. Glucose is a simple sugar with the molecular formula C₆H₁₂O₆. Glucose is the most abundant monosaccharide, a subcategory of carbohydrates. “Glucose” and “Dextrose” are often used interchangeably. Formally known as Dextrose Monohydrate or D-Glucose, dextrose is the most common type of glucose.

Nitrogen source: Nitrogen sources are nutrients that can provide a source of nitrogen in microbial cells and metabolites, including but not limited to inorganic nitrogen sources (such as ammonium salts, ammonia, nitrates, and urea), and organic nitrogen sources (such as soy flour, peanut cake, peptone, beef extract, and yeast extract). Nitrogen source involves in the biochemical synthesis of nucleic acids, lipids, and proteins in the cells.

Peptone: Peptone is a protein derivative that is formed by the partial hydrolysis of proteins under acidic conditions. It is not coagulated by heat and is soluble in water. Peptone contains polypeptides and amino acids, and it provides a readily available source of nitrogen and minerals for the growth of bacteria. Peptone is a product of the incomplete fermentative hydrolysis of protein. In an embodiment, meat peptone has been used as a nitrogen source. Meat Peptone is a highly nutritious enzymatic digest of meat and may be used as an ingredient in culture media.

Peptone is of differing source, for example, but not limited to casein, gelatin, meat, soy and yeast. Gray et al., 2008 states that “The class of the peptone and the source or manufacturer are clearly significant factors in the performance of the medium used.” (Journal of applied microbiology, 104(2), 554-565).

Meat peptones were made from enzymatic digestion of animal tissue. Therefore, characteristic biochemical property of meat peptone depends on type of tissue, which result in affecting the performance of the medium with respect to their support for microbial growth.

Other peptones made from animal sources were casein and gelatin. Casein peptone is enzymatic digestion of casein. Therefore, it is more defined compared to meat peptone.

Gray et al., 2008 states “Gelatin is a more defined substrate compared with other peptones: it is produced by boiling collagen, isolated from animal skin, bones and connective tissues and subsequent pancreatic digestion. Gelatin-based peptones produced consistently poor generation times and yields with all bacteria; analyses showed low tyrosine content relative to other peptones.” (Journal of applied microbiology, 104(2), 554-565).

Soy peptones are prepared by papain digestion of Carica papaya.

Gray et al., 2008 observed that “Casein-based broth buffered peptone water (BPW) presented a diverse set of g values irrespective of the bacterial species”, whereas “Gelatin peptones gave the poorest mean generation times.” “Yeast as a peptone source for Gram-negative bacteria generated a consistently high quality of growth with no significant difference within the group with respect to g values.” “Therefore, the inclusion of an unsuitable peptone as a constituent of the pre-enrichment medium may fail to recover the organism, which in subsequently favorable conditions, replicates and results in a bacterial population capable of human infection.” (Journal of applied microbiology, 104(2), 554-565).

In an embodiment, the peptone is meat peptone.

Beef extract: Beef Extract described herein is derived from infusion of beef and provides an undefined source of nutrients. Beef Extract is not exposed to the harsh treatment used for protein hydrolysis, so it can provide some of the nutrients lost during peptone manufacture. Beef Extract is a mixture of peptides and amino acids, nucleotide fractions, organic acids, minerals, and some vitamins. It complements the nutritive properties of peptone by contributing minerals, phosphates, energy sources, and those essential factors missing from peptone. In an embodiment, beef extract has been used as a nitrogen source.

Casein Hydrolysate: Casein hydrolysate described herein are well known food ingredients and are commonly prepared by hydrolyzing a casein substrate, typically sodium caseinate, with a food grade proteolytic/peptideolytic preparation to a degree of hydrolysis (DH) of 20% DH or greater. In an embodiment, casein hydrolysate is used as an nitrogen source.

Yeast Extract: Yeast extract is the product after removing yeast cell wall and insoluble molecule through completely autolysis by its own hydrolytic enzyme after plasmolysis. In the production process, the degree of hydrolysis of the yeast's own protein is strictly controlled to meet different application requirements. Finally, the yeast extract is concentrated to a semi-pasty, pasty product, oil-embedded, microcapsuled and micronized powdery products to meet different requirements. In an embodiment, yeast extract is used as an nitrogen source.

Buffer: Buffers described herein are molecules that donate or accept protons to resist changes in pH as acids or bases are added to the solution. A buffer consists of a weak acid and its conjugate base, or a weak base and its conjugate acid. It helps to keep any solution stable with regard to pH changes. Any buffer may be used, with the proviso that the resulting buffered solution is useful to practice the methods disclosed herein. A buffered solution can be varied as appropriate by one skilled in the art.

HEPES: N-(2-hydroxyethyl) piperazine-N′-(2-ethanesulfonic acid). In an embodiment, HEPES has been used as a buffer.

PIPES: piperazine-N,N′-bis(2-ethanesulfonic acid) In an embodiment, PIPES has been used as a buffer.

MOPS: 3-(N-morpholino) propanesulfonic acid. In an embodiment, MOPS has been used as a buffer.

Dibasic Phosphate solution: Dibasic Phosphate solution is the solution prepared by using Sodium and/or Potassium Dibasic phosphate. Generally, Dibasic and monobasic phosphate solution are prepared to prepare Phosphate buffer. In an embodiment, dibasic phosphate solution is Sodium Dibasic phosphate. Sodium phosphate dibasic solution is a reagent with very high buffering capacity widely used in molecular biology, biochemistry, and chromatography. Sodium phosphate dibasic is highly hygroscopic and water soluble. It is used in conjunction with Sodium monobasic Phosphate in the preparation of biological buffers.

Phosphate Buffer: Phosphate buffer described herein is a triprotic acid, prepared from a dibasic and monobasic phosphate solution. In an embodiment, phosphate buffer is used during pH range about 5.8 to about 7.4. A simple phosphate buffer is used ubiquitously in biological experiments, as it can be adapted to a variety of pH levels, including isotonic. Phosphate buffer is highly water soluble and has a high buffering capacity.

Monobasic Phosphate solution: Monobasic Phosphate solution is the solution prepared by using Sodium and/or Potassium monobasic phosphate.

Cysteine: Cysteine (Cys or C) is a semi-essential proteinogenic amino acid with the formula HOOC—CH—(NH2)-CH2-SH. The thiol side chain in cysteine often participates in enzymatic reactions as a nucleophile. Cysteine is a reduced form and cystine is its oxidized dimer form and has the formula (SCH₂CH(NH₂)CO₂H)₂. In an embodiment, cysteine is used in the growth media.

Sulphur containing amino acid: The term “sulphur-containing amino acid” as used herein refers to any amino acid, natural or synthetic, containing sulphur in any form, including, but not limited to, sulphydryl groups or disulphide bonds. Sulphur-containing amino acids suitable for the present invention include, but not limited to, cysteine, cystine, methionine, and their respective derivatives and synthetic analogues. Compositions according to the present invention may, in particular embodiments, comprise in addition to the protein hydrolyzate, sulphur-containing compounds such as amino acids or sulphur-containing salts which may supplement the amount of sulphur in the composition to achieve the percentages (by weight) set for the above.

Ferric pyrophosphate: The term is used interchangeably with Iron pyrophosphate. It is an inorganic chemical compound with the formula Fe₄(P₂O₇)₃ having iron in +3 state. Ferric pyrophosphate has a molecular weight of 745.25. In an embodiment, it is of ACS grade or any other commercial grade.

Blood: Blood is a body fluid in mammals such as humans and other animals that delivers necessary substances such as nutrients and oxygen to the cells and transports metabolic waste products away from those same cells. It's composition include nucleated or non-nucleated cells, nutrients, vitamins, proteins, glucose, mineral ions, hormones, gases etc. In an embodiment, blood is any mammalian blood and preferably, it is a sheep or an ox blood. The hemoglobin present in blood is collected as a 2% solution is often used in media at a 1%-10% concentration.

Francisella: Francisella is a genus of pathogenic, Gram-negative bacteria. They are small coccobacillary or rod-shaped, nonmotile organisms, which are also facultative intracellular parasites of macrophages. Strict aerobes, Francisella colonies bear a morphological resemblance to those of the genus Brucella. Described herein include all the species, varieties and subtypes of Francisella but not limited to F. tularensis, F. novicida, F. hispaniensis, F. persica, F. noatunensis, F. philomiragia, F. halioticida, F. endociliophora, F. guangzhouensis, F. piscicida. In an embodiment, the present invention relates to F. tularensis. Francisella tularensis is the bacterium that causes Tularemia, a disease that can be fatal if not detected and treated with appropriate antibiotics. The symptoms of Tularemia “could include sudden fever, chills, headaches, muscle aches, joint pain, dry cough, progressive weakness, and pneumonia”, this information can be found at the CDC website. It is on the Center for Disease Control and Prevention (CDC) list of possible bacteria that has potential as a biological warfare weapon. The CDC has developed a list of possible pathogens that may be used as weapons of mass destruction. Francisella tularensis has been listed in Category A of possible diseases and agents. Those diseases and agents in Category A are considered a high risk to national security because they “can be easily disseminated or transmitted from person to person; result in high mortality rates and have the potential for major public health impact; might cause public panic and social disruption; and require special action for public health preparedness”, as quoted at the CDC website. A close relative, F. novicida, is not pathogenic for humans but retains mouse virulence allowing manipulation under BSL-2 conditions.

Osmotic stress: The term “osmotic stress” or “osmotic shock” is interchangeably used. Osmotic shock or osmotic stress is physiologic dysfunction caused by a sudden change in the solute concentration around a cell, which causes a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. All organisms have mechanisms to respond to osmotic shock, with sensors and signal transduction networks providing information to the cell about the osmolarity of its surroundings; [2] these signals activate responses to deal with extreme conditions. Although single-celled organisms are more vulnerable to osmotic shock, since they are directly exposed to their environment, cells in large animals such as mammals still suffer these stresses under some conditions. Current research also suggests that osmotic stress in cells and tissues may significantly contribute to many human diseases. For example: In eukaryotes, calcium acts as one of the primary regulators of osmotic stress. Intracellular calcium levels rise during hypo-osmotic and hyper-osmotic stresses.

Regulation: Regulation herein described to regulate or to keep within the determined limits of the parameters which helps the bacteria or the microbe to thrive in a particular environment condition. In an embodiment, it is the regulation of pH or osmotic stress etc.

Thiamine: Thiamine is a vitamin and can be used interchangeably with Vitamin B1. In some embodiments, the growth medium contains at least about 5 mg/L thiamine or a biosynthetic precursor thereof. Thiamine is a colorless organosulfur compound with a chemical formula C12H17N4OS. Its structure consists of an aminopyrimidine and a thiazolium ring linked by a methylene bridge. In an embodiment, thiamine includes its derivatives and analogous.

Spermine: Spermine is a polyamine involved in cellular metabolism that is found in all eukaryotic cells. The precursor for synthesis of spermine is the amino acid ornithine. It is an essential growth factor in some bacteria as well. A spermine molecule with one modification (e.g., the addition of a ligand for a cellular receptor, a fatty acid, a cholesterol, a linker, or a PEG).

Selective Growth medium: It is a media based on either complex or defined media supplemented with growth-promoting or growth-inhibiting additives. The additives may be species- or organism-selective (e.g., a specific substrate, or an inhibitor such as cyclohexamide (artidione), which inhibits all eukaryotic growth and is typically used to prevent fungal growth in mixed cultures). In an embodiment, antibiotics are used for the selective growth medium.

Non-selective Growth medium: Non-selective Growth media are intended to cultivate microorganisms in order to multiply them. It is a simple culture medium without a selection agent that will allow the growth of microorganisms in an undifferentiated way. The non-selective media can be basic or enriched depending on the organism to be cultivated because some require more elements to be able to multiply. They contain all the elements that most microorganisms need to grow.

Antibiotic: In general, the term is used interchangeably with “antibacterial substance” because bacteria are the most common target microorganisms. “Antibiotic” or “antibacterial substance” means a compound capable of reducing the growth or viability of microorganisms. Antibiotics can be composed of either organic or inorganic compounds and can include bioactive molecules produced by the immune system. Antibiotic cell killing activity is defined in relative terms, e.g., compared to controls (untreated cells or cells treated with known antibiotics), but quantitative terms (e.g., cell killing) (Unit/ng substance). In general, antibiotic activity will be initially observed for a given substance as a general phenomenon, for example, as a reduction in cell growth or survival. Then the substance is typically called an antibiotic, even if the MOA (mode of action) or quantitative efficacy is unknown. Different types of antibiotics include Penicillins such as but not limited to penicillin and amoxicillin; Cephalosporins such as cephalexin (Keflex); Macrolides such as erythromycin (E-Mycin), clarithromycin (Biaxin), and azithromycin (Zithromax); Fluoroquinolones such as ciprofolxacin (Cipro), levofloxacin (Levaquin), and ofloxacin (Floxin); Sulfonamides such as co-trimoxazole (Bactrim) and trimethoprim (Proloprim); Tetracyclines such as tetracycline (Sumycin, Panmycin) and doxycycline (Vibramycin); Aminoglycosides.

Cyclic peptide antibiotic: Cyclic peptides are polypeptide chains which contain a circular sequence of bonds. This can be through a connection between the amino and carboxyl ends of the peptide, for example in cyclosporin; a connection between the amino end and a side chain, for example in bacitracin; the carboxyl end and a side chain, for example in colistin; or two side chains or more complicated arrangements, for example in amanitin. Many cyclic peptides have been discovered in nature and many others have been synthesized in the laboratory. Their length ranges from just two amino acid residues to hundreds. In nature they are frequently antimicrobial or toxic; in medicine they have various applications, for example as antibiotics and immunosuppressive agents. Antibiotic contains a cyclic peptide nucleus. In an embodiment, the cyclic peptide antibiotics are hut not limited to polymyxin and/or colistin, Amphotericin, Cefepime, Cycloheximide, Vancomycin.

Polymyxin: Polymyxin are closely related antibiotics produced by strains of Paenibacillus polymyxa and related microorganisms or synthetically produced. These cationic drugs are relatively simple peptides with a molecular weight of about 1000. Polymyxins such as polymyxin B are decapeptide antibiotics, that is, composed of 10 aminoacyl residues. They are bactericidal and are particularly effective against gram-negative bacteria such as E. coli and other species of intestinal bacteria, Pseudomonas, Acinetobacter baumannii, and the like. However, polymyxins have serious side effects including nephrotoxicity and neurotoxicity. Therefore, these drugs have been limited in their use as therapeutic agents due to their high systemic toxicity. Polymyxins B and E (also known as colistin) are used in the treatment of Gram-negative bacterial infections. They work mostly by breaking up the bacterial cell membrane. They are part of a broader class of molecules called nonribosomal peptides.

Amphotericin: Amphotericin B is an antibiotic and antifungal whose molecule is produced naturally by Streptomyces nodosus or synthetically produced. Two chemical forms exist: the Amphotericin A, without clinical application and with macrolide chemical configuration, and Amphotericin B (AmB). Its name is originated from the amphoteric properties of the chemical agent. AmB is a member of a family of nearly 200 polyene macrolide antibiotics, whose structure was unambiguously determined in 1970 through X-ray crystallography studies. In an embodiment, it is added in mg/L.

Refrigeration: Refrigeration here means archiving or storing or cooling the thing to lower and/or maintain its temperature below the ambient one (while the removed heat is rejected at a higher temperature). Refrigeration is usually done at temperature as below as about 10° C., about 9° C., about 8° C., about 7° C., about 5° C., about 4° C., about 3° C., about 2° C., about 1° C., or below.

Sterilization: As used herein, the terms “sterilize” “sterilizing” and “sterilization” refers to any process that removes, kills, or deactivates all forms of life (in particular referring to microorganisms such as fungi, bacteria, spores, unicellular eukaryotic organisms such as Plasmodium, etc.) and other biological agents like prions present in a specific surface, object or fluid, for example food or biological culture media. Sterilization can be achieved through various means, including heat, chemicals, irradiation, high pressure, and filtration. Sterilization is distinct from disinfection, sanitization, and pasteurization, in that those methods reduce rather than eliminate all forms of life and biological agents present. After sterilization, an object is referred to as being sterile or aseptic.

Autoclave: The term, “autoclave”, “autoclaved” or like should be interpreted broadly. An autoclave is a machine used to carry out industrial and scientific processes requiring elevated temperature and pressure in relation to ambient pressure/temperature. Autoclaves are used in medical applications to perform sterilization and in the chemical industry to cure coatings and vulcanize rubber and for hydrothermal synthesis. Industrial autoclaves are used in industrial applications, especially in the manufacturing of composites. Many autoclaves are used to sterilize equipment and supplies by subjecting them to pressurized saturated steam at about 121° C. (250° F.) or at higher temperature for around 15-20 minutes depending on the size of the load and the contents.

Filtration: Term such as “filter”, “filtered”, “filtration” or any like terms should be considered broadly. Filtration is used to separate particles and fluid in a suspension, where the fluid can be a liquid, a gas or a supercritical fluid. Depending on the application, either one or both of the components may be isolated. Filtration, as a physical operation enables materials of different chemical composition to be separated. A solvent is chosen which dissolves one component, while not dissolving the other. By dissolving the mixture in the chosen solvent, one component will go into the solution and pass through the filter, while the other will be retained.

Inhibitor: As used herein, the term “inhibit” or “inhibition” means the reduction or prevention of growth of mutant, subtype, variety of any fastidious microorganism. Inhibition includes slowing the rate of growth of microorganism. The growth rate can be reduced by about 20%, about 30%, about 40%, about 50%, about 60%, about 70%, about 80%, about 90%, about 100%, about 125%, about 150% or more compared to a control or untreated microorganism of the same type. Inhibition also means a reduction in the size of the colony of microorganisms. Reduction is size of colonies could be at least 10%, at least 20%, at least 30%, at least 40%, at least 50%, at least 60%, at least 70%, at least 80%, at least 90%, up to and including 100% when compared to a control or untreated microorganism of the same type.

MIC: This term is interchangeably used with ‘minimal inhibitory concentration’. Minimum inhibitory concentrations (MICs) are defined as the lowest concentration of an antimicrobial substance or antibiotic that will inhibit the visible growth of a microorganism after the incubation of the microorganism with the antimicrobial substance or antibiotic for a specific period of time. In an embodiment, MIC tests of different Francisella strains are conducted.

Disk Diffusion: The disk diffusion method (DDM) is classified as an agar diffusion method (ADM) because the substance e.g., antibiotic to be tested diffuses from its reservoir through the agar medium seeded with the test microorganism. Generally, the reservoir is a filter paper disk, which is placed on top of an agar surface. If tested substances are microbiologically active, an inhibition zone develops around the filter paper disk after incubation. The diameter of the inhibition zone properly describes the antimicrobial potency of substance or antibiotics. In an embodiment, Disk diffusion is a susceptibility test used to be performed on the isolates for the following antimicrobials but not limited to penicillin, amoxicillin, erythromycin, gentamicin, clindamycin, ciprofloxacin, cephalexin, chloramphenicol, tetracycline, oxytetracycline, vancomycin, cefotetan, moxifloxacin, polymyxin and/or colistin, amphotericin, cefepime, cycloheximide, and rifampin. Disk diffusion test is also known as Kirby-Bauer test, disc-diffusion antibiotic susceptibility test, disc-diffusion antibiotic sensitivity test and KB test.

Gram-negative bacterium: Gram-negative bacteria are bacteria that do not retain the crystal violet stain used in the gram-staining method of bacterial differentiation and have inner cell wall. In an embodiment, Francisella is a Gram-negative bacterium.

Zoonotic disease: Zoonotic disease is defined herein broadly as a disease that spreads from animals to people.

Infectious: Infectious means transmissible or communicable e.g., a disease can be infectious. In an embodiment, tularemia is an infectious disease.

Virulent: “Virulence” and “virulent strains” similarly have meanings extending beyond the dictionary definition of extremely infectious, malignant, or poisonous. Parasite virulence and host resistance determine how host and parasite interact in ecological time and how they co-evolve. Virulence is often defined as an increase in the host mortality rate as a result of the parasite's presence. But reduced host fecundity, parasite replication rate within the host, and several other measures have also been used. Virulence should in principle also include instances where the behavior of the host is manipulated by the parasite to increase the probability of its successful transmission and where it places the individual host at greater risk. Here the terms virulent and virulence are used in a broad sense that encompasses all of these meanings.

Model strain: A model strain is described as a strain of a living being which acts as a model for a particular purpose e.g., to study for a disease. In an embodiment, tularemia research is usually carried out first using closely related, but less virulent model strains.

Standard microbiological media: The term herein described as the microbiological media which is most generally used for the purpose.

Pathogen: A pathogen refers to a microorganism that can cause disease in its host. Non-limiting examples of a pathogen include a prion, a virus, a bacterium, a fungus, a protozoan, a helminth, and a parasite.

Chamberlain's defined medium: A chemically defined medium prepared and provided by Chamberlain (1965), which adequately supported growth of a vaccine strain of but not limited to Pasteurella tularensis, Francisella.

NDM: The simplified version of Chamberlain's defined medium to grow Francisella is referred to as NDM.

Minimal media: A defined medium that has just enough ingredients to support growth is called a “minimal medium”. The number of ingredients that must be added to a minimal medium varies enormously depending on which microorganism is being grown. Minimal media are those that contain the minimum nutrients possible for colony growth and are often used by microbiologists and geneticists to grow “wild-type” microorganisms. Minimal media can also be used to select for or against recombinants or exconjugants. In various embodiments a minimal media may be developed and used to grow microorganisms that contains the minimal necessities or without the presence of a mixture of undefined agents for growth of the wild-type. In an embodiment, a medium may contain inorganic salts, a carbon source, and water. These minimal medias may also have limited supplementation of vitamin mixtures including but not limited to biotin, vitamin B12 and derivatives of vitamin B12, thiamin, pantothenate and other vitamins. In another embodiment, minimal media may also have limited simple inorganic nutrient sources containing phosphate, sulfate, and total nitrogen.

Glycerol stocks: The addition of glycerol stabilizes the frozen bacteria, preventing damage to the cell membranes and keeping the cells alive. A glycerol stock of bacteria can be stored stably at −80° C. for many years. In an embodiment, the microbial cultures used to prepare glycerol stock cultures by vigorous mixing with an equal volume of 100% sterile glycerol, followed by freezing and storage at −80°. In an embodiment, percentage of the glycerol in microbial culture solution could vary from but not limited to about 10% v/v, about 20% v/v, about 30% v/v, about 50% v/v, about 60% v/v, about 75% v/v, about 90% v/v, about 100% v/v.

Assay: It is an analytic procedure for qualitatively assessing or quantitatively measuring the presence, growth, amount, or functional activity of a target entity (the analyte). In an embodiment, the present invention includes but not limited to disk diffusion assay.

Tryptic soy broth: Tryptic soy broth is as known in the art. Tryptic soy broth generally comprises tryptone (a pancreatic digest of casein), Soytone (a papaic digest of soybean meal) and sodium chloride, for example. Modified tryptic soy broth may further comprise dextrose, bile salts and dipotassium phosphate. Particularly the base broth is selected from the group consisting of tryptone, nutrient broth, L-broth, gram negative broth, peptone, tryptic soy broth, tryptic soy broth with yeast and modified tryptic soy broth. More particularly the base broth is selected from the group consisting of peptone, tryptic soy broth, tryptic soy broth with yeast and modified tryptic soy broth.

Generation of Francisella Lawns: As described herein Francisella lawn is a term used to describe the appearance of the colonies when all the individual colonies on solidified media merge to form a field or mat.

Generation of Spontaneous mutants on agar: Mutations in an organism's genome can arise spontaneously, i.e., in the absence of exogenous stress and prior to selection. In an embodiment, spontaneous generation may lead to but not limited to antibiotic resistance colonies.

Competition assays: In an embodiment, assays may also include “competitive assays” that i) employ an immobilized capture reagent that competes with an analyte for binding to a detection reagent or ii) a detection reagent that competes with an analyte for binding to an immobilized capture reagent. In the case of the competitive assay, the presence of analyte leads to a measurable decrease in the amount of detection reagent on the binding surface.

The invention is illustrated below through various embodiments.

In an embodiment, Francisella is a facultative intracellular pathogen of phagocytic eukaryotic cells. Culturing organisms that have an intracellular step to their lifecycle is generally more difficult than extracellular pathogens. There are currently two chemically defined media that grow Francisella. One is Chamberlain's defined medium (CDM) (17) and the other is a simplified version referred to as NDM (18). While these are not technically Francisella minimal media (18), they are the best approximations available. Beyond the standard amino acids and carbon sources it is important to note that in both these media there is an iron source as well as calcium pantothenate, spermine and thiamine. While these ingredients may be added independently, they are likely present in small amounts in eukaryotic digests leading to variance in exact content of digest in describing essential media for Francisella tularensis.

In an embodiment, beyond just culturing Francisella in axenic culture, being able to isolate it from complex biological samples is yet another hurdle, as Francisella is slower growing and can be quickly over-run by other contaminating microbes in complex biological samples (28-30). The isolation of Francisella from environmental and animal samples is important for understanding the prevalence of Francisella and being able to monitor outbreaks of Francisella. Being able to isolate Francisella from environmental samples and mammalian hosts is also essential in identifying potential biothreat incidents.

In an embodiment, some ingredients are accepted to be absolutely essential. Cysteine/Cysteine (20) is determined to be essential very early after the first isolation of Francisella (21). Then thiamine was discovered as important for Francisella when studies demonstrated that boiling blood (which was later added to casein hydrolysate) in lye slowed the growth rate of Francisella growth and yet boiling it in acid did not. This was confirmed by the independent addition of thiamine after boiling which restored Francisella growth (22). While there is no definitive research showing the essential nature of spermine, it is believed to shorten the lag phase when growing Francisella (23). Many Francisella genes are known to be sensitive the presence of spermine in the culture medium (24). As spermine is present inside of all eukaryotic cells, it might signal to Francisella to regulate intracellular infection & pathogenicity genes. This theory is supported by the fact that the disruption of these spermine responsive genes leads to a significant reduction in virulence (25). Iron is also known to be critical for Francisella tularensis growth (26, 27), which is expected given its pathogenic nature.

In an embodiment, F. tularensis tularensis is classified as a Tier 1 Category A Select Agent. Francisella is a gram-negative bacterium that causes the zoonotic disease tularemia (1-3), and is classified as a Category A, Tier 1 threat agent. Due to the extremely infectious nature of fully virulent Francisella tularensis, it has to be studied at biosafety level (BSL)-3. Thus, tularemia research is usually carried out first using closely related, but less virulent model strains in order to optimize experiments before they are performed on fully virulent strains in a BSL-3 environment.

In an embodiment, the “type strain” deposited for the fully virulent (type A) Francisella tularensis tularensis at BEI resources is an avirulent less-commonly used strain of Francisella known as Francisella tularensis subsp. tularensis, strain NIH B38 (NR50, ATCC6223, FSC230 BEIResources.org) (4). This strain has lost virulence through laboratory passage and thus is a BSL2 strain. Importantly, NIH B38 is considered the type strain for highly virulent Francisella tularensis subsp. tularensis, type A biotype (5) and has been used as such in several studies (6-12). While the NIH B38 strain is genetically more similar to the fully virulent F. tularensis tularensis SchuS4 than to F. novicida U112 or F. holarctiva LVS, NIH B38 is currently seldom utilized as a model for SchuS4 as it is difficult to culture in standard broths used for other tularemia model organisms (8).

In an embodiment, some genetic differences from F. tularensis SchuS4 were noted for the NIH B38 Type strain(7, 13); however, in analysis of RD-1 (A region of difference used to genetically distinguish Francisella strains) for example, the strains were identical (13). The strain has been sequenced (BioProject PRJNA30629, www.ncbi.nlm.nih.gov/bioproject/30629). Sequence analysis suggests that the Type strain NIH B38 clusters with Type A tularemia strains identified from California, Wyoming and Nevada (14). In an embodiment, the gap was bridged between BSL-2 and BSL-3 studies by developing a novel medium capable of growing NIH B38 as well as SchuS4.

In an embodiment, while the genetic similarity and BSL-2 status support NIH B38 as an excellent choice for conducting preliminary experiments, many researchers forgo using NIH B38 in favor of other BSL-2 Francisella strains which are more distantly related to the fully virulent type A strain SchuS4 such as F. novicida U112 and F. holarctica LVS, as these microbes are much easier to culture in standard microbiological media (15, 16). Wanting to enable more researchers to use the more genetically relevant NIH B38 strain, a tractable new medium capable of growing even the most fastidious of Francisella strains was created. In order to design the optimal medium, both historic and modern studies investigating the nutritional requirements of Francisella were first examined.

In an embodiment, a medium has been developed for growth of Francisella. The medium is called as FIRE.

In an embodiment, a type of liquid growth medium that could grow the fully virulent BSL-3 strains as well as the BSL-2 type strain NIH B38 has been created. A medium that could be made cost effectively, could be made selective for Francisella, and did not contain any blood (34). Blood is expensive, must be refrigerated, must be chocolatized into the media, and expires rapidly. Others have also tried to create broths capable of supporting Francisella growth that do not include blood (35). To that end a novel medium without blood that yields rapid Francisella growth has been developed.

In an embodiment, the medium is called as FIRE (Francisella isolation, recovery, and enrichment) based on its capabilities.

In an embodiment, ingredients of FIRE medium are illustrated shown in Table 1.

TABLE 1
Solid Media Composition (Approximate formula per liter of purified H20)

| Medium | Ingredients | | | Antibiotics | | |
|---|---|---|---|---|---|---|
| GC II | Pancreatic Digest of Casein | 7.5 | g | Not Applicable | | |
| Chocolate | Sodium Chloride | 5.0 | g | | | |
| agar | Selected Meat Peptone | 7.5 | g | | | |
| | Agar | 12.0 | g | | | |
| | Corn Starch | 1.0 | g | | | |
| | Hemoglobin | 10.0 | g | | | |
| | Dipotassium Phosphate | 4.0 | g | | | |
| | IsoVitalex Enrichment | 12.0 | ml | | | |
| | Monopotassium Phosphate | 1.0 | g | | | |
| | Pyridoxol | 0.01 | g | | | |
| | Growth Factors | 0.5 | g | | | |
| Remel | Casein Peptone | 13.0 | g | Penicillin | 67.7 | mg/L |
| | Dextrose | 10.0 | g | Polymyxin B | 16.7 | mg/L |
| | Sodium Chloride | 5.0 | g | | | |
| | Yeast Extract | 5.0 | g | | | |
| | Beef Heart Infusion | 2.0 | g | | | |
| | L-Cysteine | 1.0 | g | | | |
| | Agar | 15.0 | g | | | |
| | Rabbit Blood | 5% | | | | |
| CHAB- | Beef Heart Infusion | 10.0 | g | Amphotericin | 2.5 | mg/L |
| PACCV (28) | Proteose Peptone | 10.0 | g | Cefepime | 4.0 | mg/L |
| | Dextrose | 10.0 | g | Cycloheximide | 100.0 | mg/L |
| | Sodium Chloride | 5.0 | g | Vancomycin | | |
| | L-Cysteine | 1.0 | g | Polymyxin B | 4.0 | mg/L |
| | Agar | 15.0 | g | | 8.0 | mg/L |
| | Sheep Blood | 9% | | | | |
| CHAB- | Beef Heart Infusion | 10.0 | g | Amphotericin | 2.5 | mg/L |
| CACCV | Proteose Peptone | 10.0 | g | Cefepime | 4.0 | mg/L |
| | Dextrose | 10.0 | g | Cycloheximide | 100.0 | mg/L |
| | Sodium Chloride | 5.0 | g | Vancomycin | | |
| | L-Cysteine | 1.0 | g | Colistin | 4.0 | mg/L |
| | Agar | 15.0 | g | | 8.0 | mg/L |
| | Sheep Blood | 9% | | | | |
| FIRE | Meat Peptone | 52.5 | g | Amphotericin | 2.5 | mg/L |
| | Dextrose | 10.0 | g | Cefepime | 4.0 | mg/L |
| | Sodium Chloride | 10.0 | g | Cycloheximide | 100.0 | mg/L |
| | L-Cysteine Hydrochloride | 0.05 | g | Vancomycin | | |
| | Monohydrate | | | Colistin | 4.0 | mg/L |
| | Agar | 15.0 | g | | 8.0 | mg/L |
| | Ferric Pyrophosphate | 0.25 | g | | | |
| | Sodium Phosphate Monobasic | 2.5 | g | | | |
| | Monohydrate | | | | | |
| | Sodium Phosphate Dibasic | 2.4 | g | | | |
| | Anhydrous | | | | | |

In an embodiment, the medium includes sodium chloride which is both for metabolism but also a regulator of osmotic stress; glucose as a carbon source; meat peptone as a nitrogen source but also for its inclusion of eukaryotic factors like thiamine spermine; cysteine and ferric pyrophosphate because fastidious organisms like Francisella are known to require these and finally dibasic and monobasic sodium phosphates to function as a buffering system. Other protein sources (Beef extract, Casein Hydrolysate, Yeast Extract), carbon sources (Lactose, Sucrose) and buffers (HEPES, PIPES, MOPS, Dibasic/Monobasic Potassium Phosphate) were tried but yielded less growth in the combinations prepared.

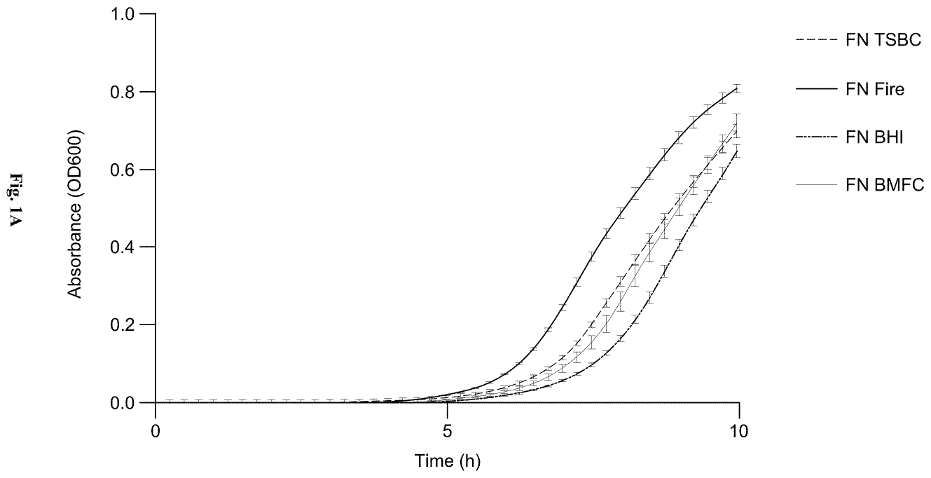

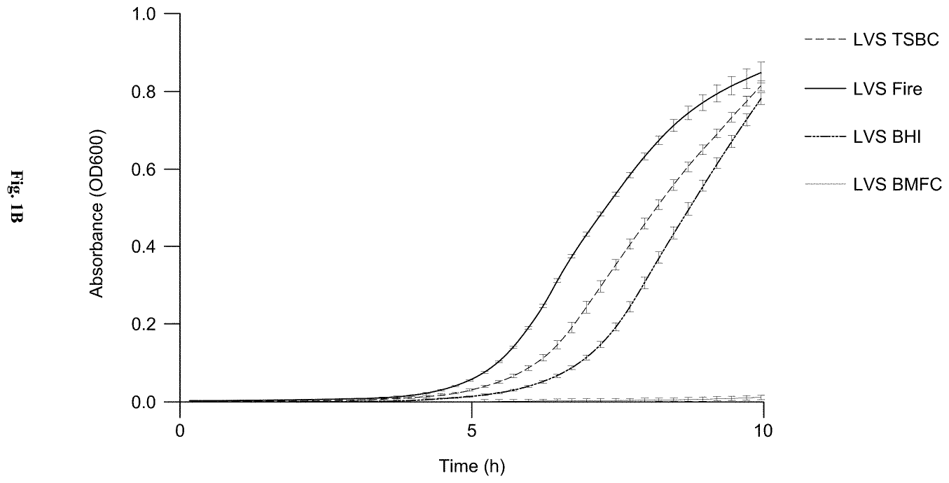

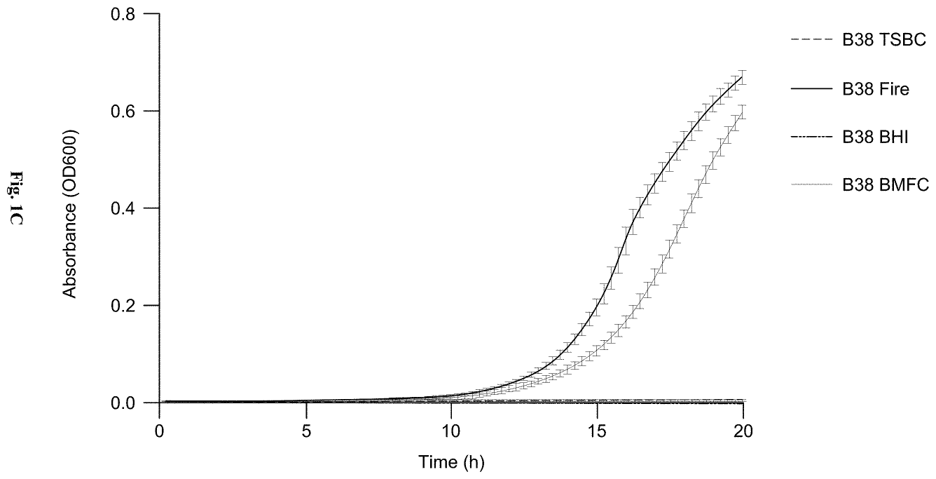

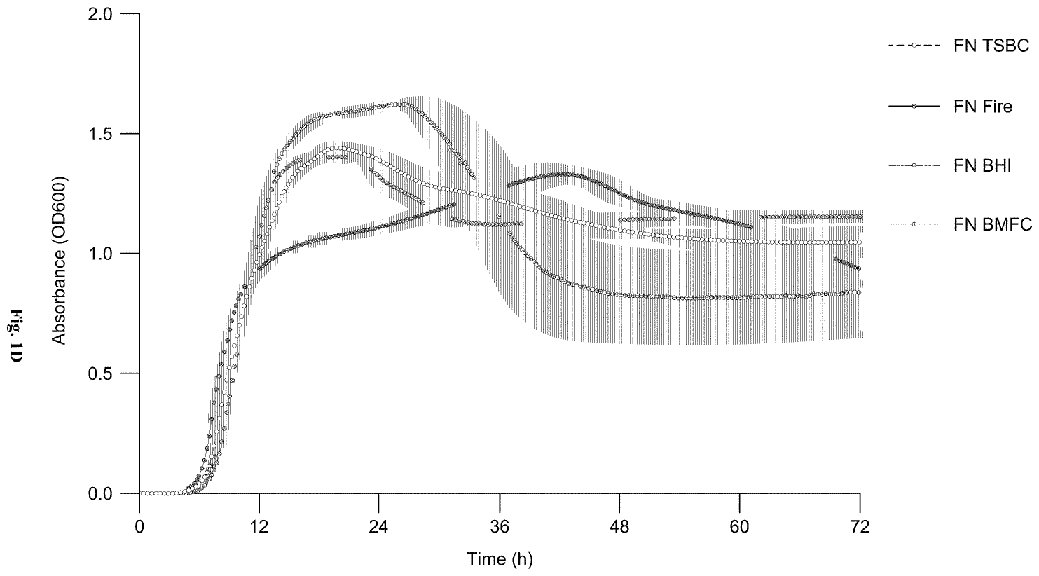

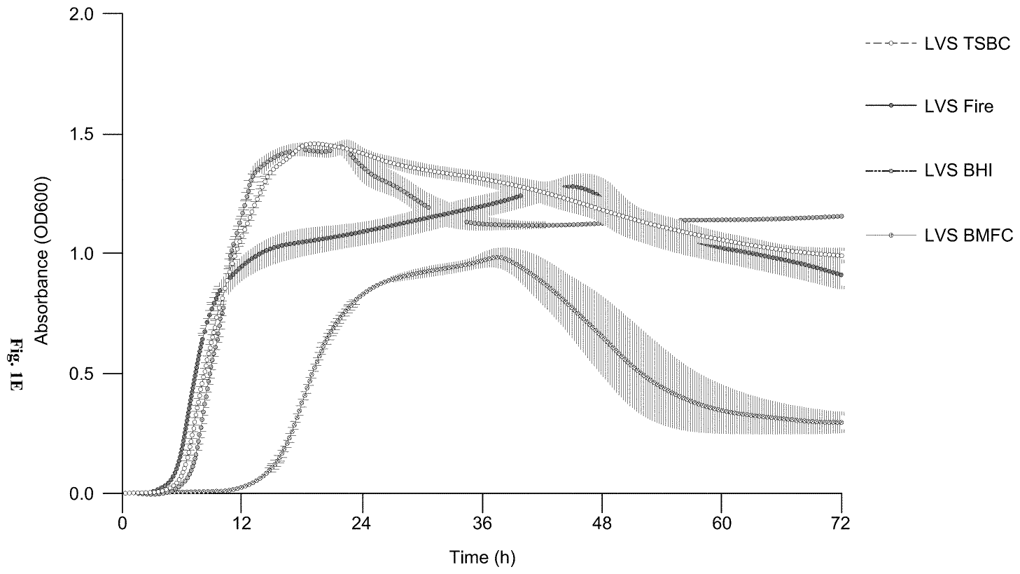

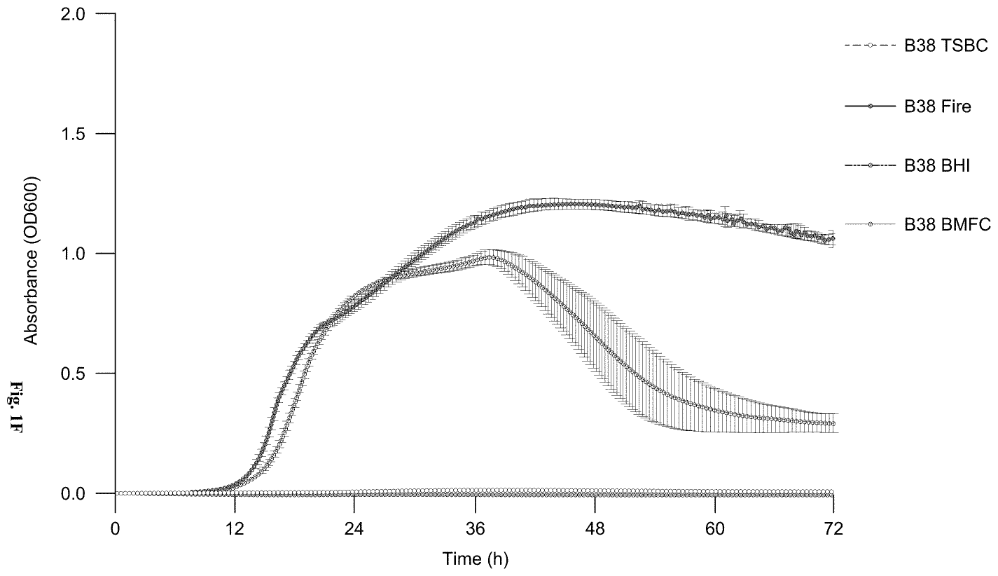

In an embodiment, in order to directly compare the utility of FIRE medium compared to established Francisella media, growth curves comparing FIRE to Tryptic Soy Broth with 0.1% (w/v) cysteine (TSBC), brain heart infusion medium (BHI), and a relatively uncommon medium BMFC which is a combination of Mueller-Hinton broth and brain heart infusion with some added cysteine and iron were performed. TSBC and BHI were chosen due to their ubiquity whereas BMFC was chosen as it was a broth that was reported to support the growth of NIH B38. We performed this testing for F. novicida, F. tularensis holarctica LVS and NIH B38 as shown in FIG. 1A to 1C.

In an embodiment, FIRE medium resulted in the fastest growth for all strains tested in this format. The full 72-hour observation period for each growth curve is shown in FIG. 1D to FIG. 1E. When grown with shaking in 5 ml of FIRE at 37° C., cultures of LVS produce turbidity overnight, cultures of F. tularensis NIH B38 produce turbidity within 36 hours and cultures of F. tularensis SchuS4 produce turbidity within 48 hours. This speed of growth for the more fastidious Francisella strains is unprecedented.

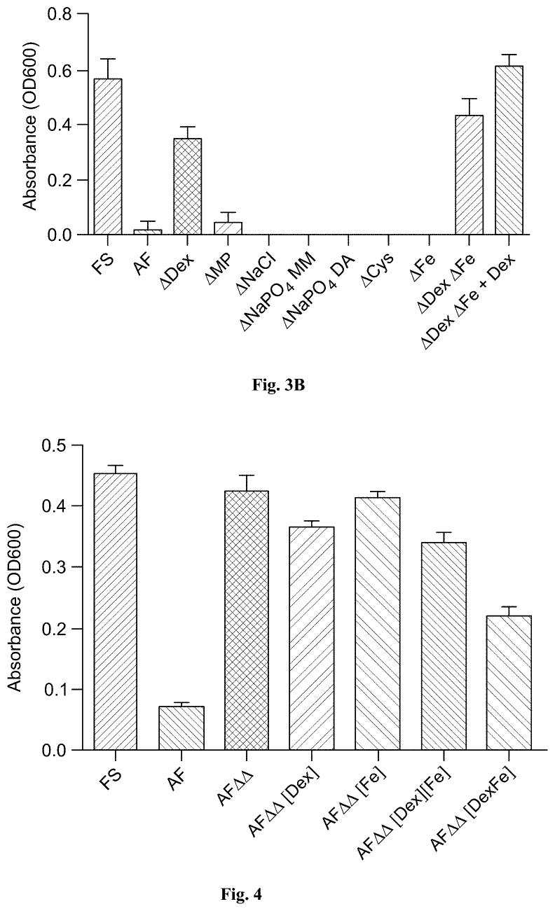

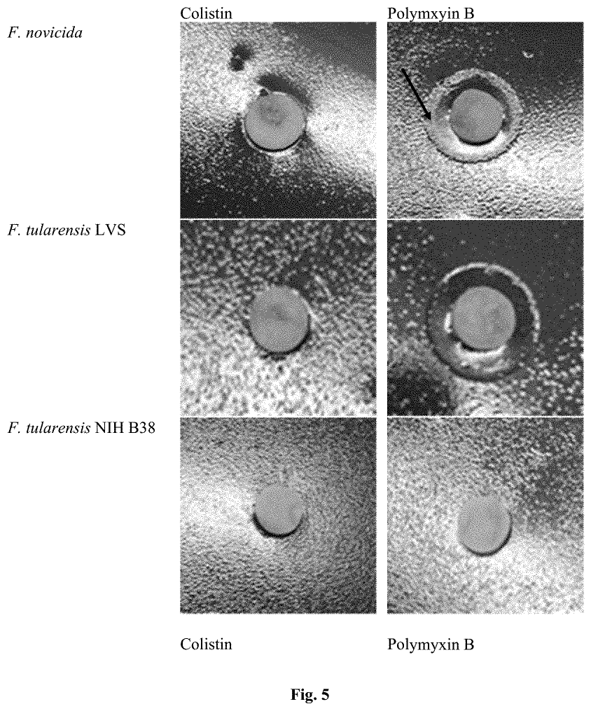

In an embodiment, during early experimentation to refine FIRE (FIG. 3B) it was discovered that autoclaved FIRE would not support F. tularensis NIH B38 growth while it did support the growth of both F. novicida and F. tularensis LVS. It has been hypothesized this autoclaving effect to be due to the destruction/sequestration of nutrients, the decrease in pH, or the accumulation of toxic byproducts.

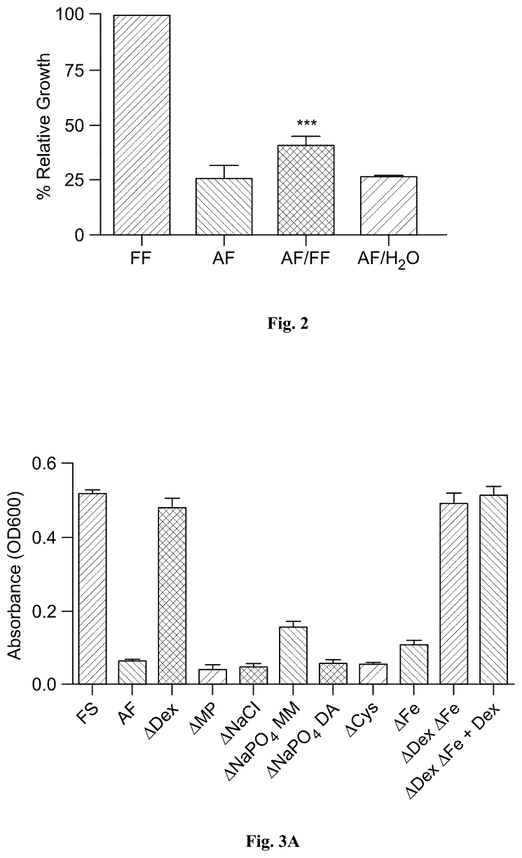

In an embodiment, to eliminate the nutritional deficit theory, 100 μl of autoclaved medium was added to 100 μl inoculated 2×FIRE medium. These conditions stimulated growth at levels of 45% relative to NIH B38 grown in filter-sterilized FIRE medium (FIG. 2). We subsequently tested the pH of autoclaved FIRE and found it to be pH 6.1 compared to the pH 6.7 of filter-sterilized FIRE medium. Using hydrochloric acid, we tested the range at with F. novicida LVS and NIH B38 could grow in FIRE medium. F. novicida grew in pH 4 after 24 hours whereas LVS became turbid after 48. Growth of NIH B38 was not witnessed at pH 4 during the 2-week observation period (Table 2).

TABLE 2
pH tolerance of Francisella strains

| pH | 4 | 5.1 | 6.1 | 6.7 |
|---|---|---|---|---|
| F. novicida | Growth | Growth | Growth | Growth |
| F. tularensis LVS | Growth | Growth | Growth | Growth |
| F. tularensis NIH B38 | No Growth | Growth | Growth | Growth |

In an embodiment, the theory was investigated that NIH B38 might be inhibited by a toxic byproduct of the autoclaving process. Caramelization products (36) and Maillard reaction products (37) for their ability to inhibit NIH B38 growth were tested. First, to test the caramelization products, using the concentration of available reactants in FIRE medium, 2× solution of dextrose was prepared which was then diluted two-fold serially across 1× filtered FIRE medium. This resulted in no growth inhibition. Then in order to test the Maillard products, the amino acid ingredients (Meat Peptone/Cysteine) and the Dextrose all at 2× concentration were autoclaved and diluted them two-fold across 1×FIRE medium. No growth of NIH B38 was observed at the highest concentration tested. Francisella novicida and LVS were subsequently tested and were able to grow in this concentration (Table 3).

TABLE 3
Maillard reaction product tolerance in Francisella strains

| % Maillard Reaction Product | 100 | 50 | 25 | 12.5 |
|---|---|---|---|---|
| F. novicida | Growth | Growth | Growth | Growth |
| F. tularensis LVS | Growth | Growth | Growth | Growth |
| F. tularensis NIH B38 | No Growth | Growth | Growth | Growth |

In an embodiment, as none of the previous attempts were able to conclusively identify the reason why NIH B38 would not grow in autoclaved FIRE medium, a single ingredient elimination strategy was chosen to pursue, where several versions of autoclaved FIRE medium were made each lacking one ingredient. Each of these solutions were then inoculated and growth was assayed. After initial testing, both ferric pyrophosphate and dextrose were left out of autoclaved FIRE medium and this solution was tested as well (FIG. 3A). This was also carried out using the fully virulent F. tularensis SchuS4 (FIG. 3B) in order to determine if this was an NIH B38 specific issue. Both strains grew well in autoclaved FIRE with the dextrose removed. Adding the dextrose in after autoclaving caused a modest growth increase over the “dextrose-removed” condition. The iron ferric pyrophosphate was only able to be removed successfully when the dextrose was also removed.

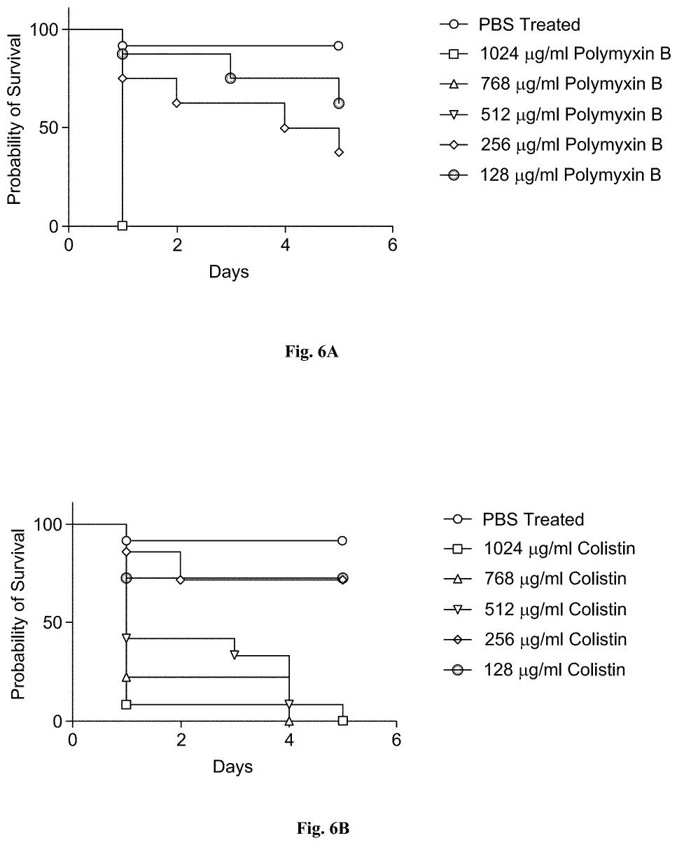

In an embodiment, in order to further test the idea that the inhibiting product was some sort of ferric-pyrophosphate-dextrose chelation compound, we grew NIH B38 in autoclaved FIRE without either dextrose or pyrophosphate and then we added back in autoclaved dextrose or autoclaved ferric pyrophosphate or a mixture of the two autoclaved separately or finally, the two autoclaved in the same bottle. The concentrations of each solution were diluted correctly to reflect the reactants that would normally be present in FIRE medium. The condition with the added in ferric pyrophosphate and dextrose that were autoclaved together yielded the lowest growth, about half that of autoclaved FIRE without dextrose and without ferric pyrophosphate (FIG. 4). Unfortunately, although the starting concentration of reactants was maintained, as we knew neither the product nor the mechanism of its creation, at this point it is impossible to compare the efficiency of the reaction produced in isolation to the efficiency in the presence of the other ingredient which may act as buffering agents or catalysts.

In an embodiment, we concluded that the inhibitory compound could most likely be made with just dextrose and ferric pyrophosphate.

In an embodiment, in order to identify this unique compound(s) we autoclaved 2× ferric pyrophosphate and 2× dextrose separately and then combined them and performed Quadrupole Time of Flight Mass Spectrometry (Q-TOF-MS) on the lyophilized sample. We then compared that to the Q-TOF-MS results of 1× dextrose and 1× ferric pyrophosphate autoclaved together. We were able to identify several unique compounds formed in autoclaved together sample (Table 4).

TABLE 4
Identification of compounds using Q-TOF-MS

| | | | | | | FDR | Log |
|---|---|---|---|---|---|---|---|
| | | Fold | | | | Adjusted p | (Adjusted p |
| Name | Synonyms | Change | precursor_mz_rt | CID | class | value | value) |
| 1- | 1.3,4,5- | 228.06 | 146.0821_0.44 | 146.08, | Miscellaneous | 0.00010109 | −3.99529 |
| Deoxynojirimycin | Piperidinetriol, 2- | | | 128.07, | | | |
| | (hydroxymethyl)-, | | | 110.07, | | | |
| | (2R,3R,4R,5S)- | | | 98.06 | | | |
| Linustatin | Propanenitrile, 2- | 53.665 | 248.1127_0.44 | 248.11, | Miscellaneous | 0.00033302 | −3.47753 |
| | [(6-O-β-D- | | | 230.1, | | | |
| | glucopyranosyl-β- | | | 212.08, | | | |
| | D- | | | 152.07, | | | |
| | glucopyranosyl)oxy]- | | | 126.0598. | | | |
| | 2-methyl- | | | 98 | | | |
| N-Acetyl- | 2-Acetamido-2- | 36.177 | 224.1132_0.44 | 224.11, | Hexoses & | 0.00048714 | −3.31235 |
| D- | deoxy-D-galactitol | | | 206.1, | Derivatives | | |
| galactosaminitol | | | | 188.09, | | | |
| | | | | 146.08 | | | |
| Streptomycin | D-Streptamine, O- | 28.813 | 246.097_0.4 | 246.09, | Miscellaneous | 0.00021636 | −3.66482 |
| A | 2-deoxy-2- | | | 204.05, | | | |
| | (methylamino)-α-L- | | | 186.01 | | | |
| | glucopyranosyl-(1->2)- | | | | | | |
| | O-5-deoxy-3- | | | | | | |
| | C-formyl-α-L- | | | | | | |
| | lyxofuranosyl-(1->4)- | | | | | | |
| | N1,N3- | | | | | | |
| | bis(aminoiminomethyl)- | | | | | | |
| Lauryl | Dodecyl sulfate | 13.666 | 265.1467_6.74 | 265.14, | Fatty acids | 0.0015411 | −2.81217 |
| sulfate | | | | 96.95 | & | | |
| | | | | | Derivatives | | |
| D-Gluconic | Pentahydroxycaproic | 8.5925 | 195.0498_0.44 | 195.04, | Hexoses & | 0.0016762 | −2.77567 |
| acid | acid | | | 129.09, | Derivatives | | |
| | | | | 99.00, | | | |
| | | | | 87.00, | | | |
| | | | | 75.00, | | | |
| | | | | 71.01 | | | |
| myo- | | 4.6885 | 179.0538_0.47 | 179.05, | Hexoses & | 0.0033667 | −2.47280 |
| Inositol | | | | 161.04, | Derivatives | | |
| | | | | 125.02, | | | |
| | | | | 71.01, | | | |
| | | | | 59.01 | | | |
| D- | | 4.2535 | 165.0361_0.46 | 165.03, | Pemtoses | 0.0071549 | −2.14540 |
| Arabinonic | | | | 129.01, | & | | |
| acid | | | | 87.00, | Derivatives | | |
| | | | | 75.00, | | | |
| | | | | 59.01 | | | |
| Butyrolactone | 3 -Furancarboxylic | 0.075563 | 185.0812_1.87 | 185.08, | Miscellaneous | 0.0044455 | −2.35208 |
| 3 | acid, tetrahydro-4- | | | 167.06, | | | |
| | methylene-5-oxo-2- | | | 149.06, | | | |
| | propyl-, (2R,3S)- | | | 139.07, | | | |
| | rel- | | | 93.06 | | | |

In an embodiment, these include formation of three potentially antimicrobial compounds and a detergent. The three antibiotic compounds included 1-Deoxynojirimycin, which was found at 228 fold above non-autoclaved FIRE, Linustatin, which was enriched 54 times above non autoclaved FIRE and Streptomycin, found at 29 fold enriched over the regular FIRE media. In addition, the detergent Lauryl sulfate was found at 14 times higher than in the non-autoclaved FIRE media. Several other compounds were also statistically different, including N-Acetyl-D-galactosaminitol (36-fold enriched), D-Gluconic acid (9-fold enriched), myo-Inositol (5-fold enriched) and D-Arabinonic acid (4-fold enriched).

In an embodiment, the three antibiotics and the detergent were tested and found to have significant growth-inhibition effect on Francisella when tested in MIC format using FIRE medium. Thus, the formation of these compounds could be significantly contributing to the growth inhibition observed in our autoclaved FIRE medium.

In an embodiment, these compounds were tested in MIC format using Fire Medium against F. novicida, F. holarctica LVS and F. tularensis NIH B38 (Table 6). The MIC of Streptomycin against NIH B38 under standard conditions is 0.25 μg/ml (38) and that of LVS is 0.38 μg/ml (39).

In an embodiment, in isolating Francisella from environmental samples (28-30), or when plating from organ homogenates from infected animals to determine the number of colony forming units (30, 40), it is critical to use Francisella selective media which contains antibiotics to control other organisms but allow Francisella growth. Inspired by selective Francisella media developed previously (29, 41, 42), we first crafted our major counter selective bacterial agent to be a polymyxin. Performing both MIC and Disk Diffusion based antibiotic resistance testing we determined that all the strains of Francisella tested had equal or greater resistance to colistin (Polymyxin E) than to Polymyxin B, which further encouraged the development of selective FIRE with this antibiotic.

In an embodiment, Francisella is highly resistant to polymyxin B as well as colistin (polymyxin E) in the form of colistin sulfate (43). Li et al. reported a MIC of Francisella novicida to be 38 ug/ml, and showed that mutants in the lpxD2 gene caused a significant increase in resistance to Polymyxin B to 512 ug/ml (44). These experiments were performed using an E-test strip system. “The minimum inhibitory concentration (MIC) for the ΔlpxD1mutant was ˜2.5-fold (14 μg/mL) less than that of the wild type F. novicida (38 μg/mL) whereas the ΔlpxD2mutant was ˜13.5-fold (512 μg/mL) more resistant.” LPS remodeling was proposed as the reason for this increased resistance due to the lpxD mutants. Other researchers and our experiments below report a much higher level of endogenous resistance of Francisella novicida to polymyxin B (Table 5).

TABLE 5
Previous Polymyxin Francisella inhibition studies

| | Resistance | | | | | | | | | |
|---|---|---|---|---|---|---|---|---|---|---|
| | to | | | | | | | | | This |
| | Polymyxin | | Resistance to | | Kirby Bauer | | Kirby Bauer | | This study | study |
| Bacteria | B | | Colistin | | Polymyxin B | | Colistin | | Polymyxin B | Colistin |
| Francisella | 38 ug/ml | (76) | 1024 ug/ml | (43) | 19 mm | (78) | | | 256 | Right |
| novicida | 650 ug/ml | (77) | | | | | | | | around |
| | 800 ug/ml | (68) | | | | | | | | 1024 |
| Francisella | | | | | 6 mm (79) | | 0 mm | (81) | 256 | Over |
| tularensis | | | | | 30 mm (80) | | | | | 1024 |
| holarctica | | | | | other method | | | | | |
| Live | | | | | 16 mm (81) | | | | | |
| Vaccine | | | | | | | | | | |
| Strain | | | | | | | | | | |
| Francisella | | | | | | | | | Over 1024 | Over |
| tularensis | | | | | | | | | | 1024 |
| NIH B38 | | | | | | | | | | |
| Francisella | | | | | 10 mm | (78) | | | . | |
| tularensis | | | | | | | | | | |
| tularensis | | | | | | | | | | |
| Schu4 | | | | | | | | | | |
| Francisella | | | 256 ug/ml | (43) | | | | | 1024 | Over |
| philomiragia | | | | | | | | | | 1024 |

In an embodiment, the selective medium would contain the cyclic peptide antibiotic (polymyxin or colistin) to which Francisella was most resistant, as this would have the least negative effect on the growth of Francisella. To determine whether Francisella was more resistant to colistin or polymyxin B we conducted MIC tests of different Francisella strains. Francisella MIC testing has generally been conducted in modified Mueller Hinton broth (26, 32) in an effort to match the testing parameters of other microorganisms following the CLSI protocol (45). We used the standard broth dilution method following CLSI protocols and have found the MICs of Francisella strains to polymyxin B to vary from 256 μg/ml to over 1024 μg/ml. (Table 6).

TABLE 6
MICs of colistin and polymyxin for Francisella strains

| MIC | F. novicida | F. tularensis LVS | F. tularensis NIH B38 |
|---|---|---|---|
| Polymyxin B | 256 μg/ml | 256 μg/ml | >1024 μg/ml |
| Colistin | 1024 μg/ml | >1024 μg/ml | >1024 μg/ml |

In an embodiment, the difference in our MIC results for F. novicida verses Li et al. (44) could be due to their E-test strip method being done on agar vs. the liquid culture format of the MIC, as agar can affect the diffusion of the antibiotics in some cases (10, 33).

In an embodiment, F. tularensis infections are clinically treated with ciprofloxacin and aminoglycoside antibiotics. Francisella bacteria are resistant to some antibiotics such as beta-lactams (46) and cyclic peptide antibiotics like polymyxin. In addition, drug resistance to conventional antibiotic treatments is beginning to emerge in Francisella (46-48) and spontaneous resistance to other antibiotics has been observed in the genus (10, 33, 48).

In an embodiment, in order to confirm the results of MIC testing we used disk diffusion method to expose F. novicida, F. tularensis LVS, and F. tularensis NIH B38 to very high levels of polymyxin B and colistin for several days. Zones of inhibition were only seen when F. novicida and F. tularensis LVS were grown with a polymyxin B disk and not with a colistin disk, suggesting that the MIC for colistin is higher than the concentration applied to the disk. Polymyxin B resistant F. novicida colonies were seen emerging within the zone of inhibition, suggesting that further resistant colonies can spontaneously develop against these cyclic peptide antibiotics. Three of these colonies were cultured and subsequently tested to determine their MIC of Polymyxin B. All three had an MIC of 512 μg/ml, which is higher than we found for the parental F. novicida strain at 256 μg/ml, suggesting that they had increased polymyxin resistance. The mechanism for this resistance was not determined, but maybe due to alterations in the LPS as suggested by Ernst et al.

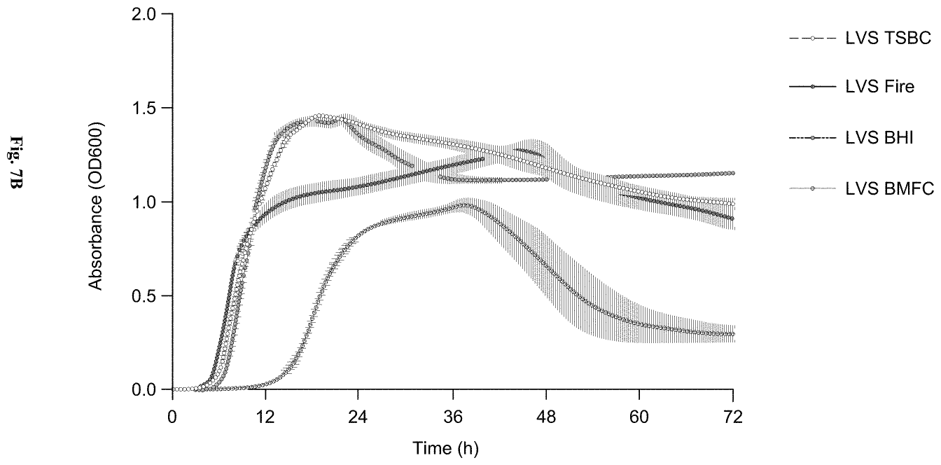

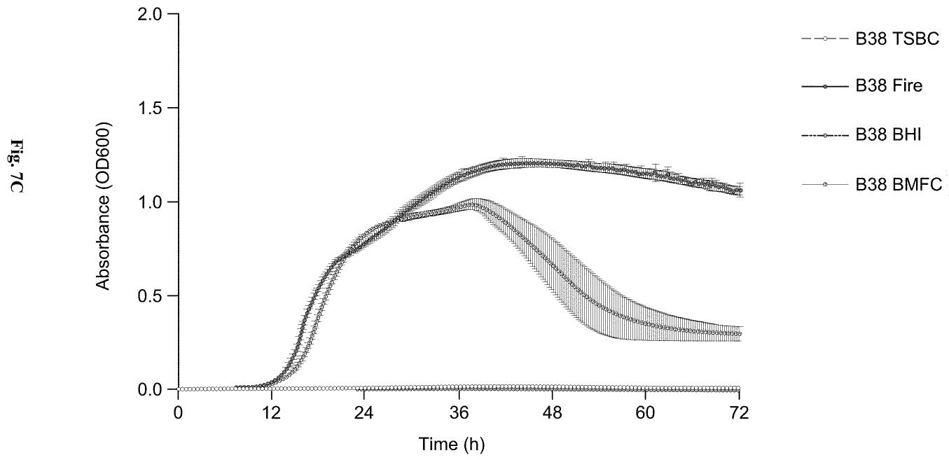

In an embodiment, we sought to further confirm the relative efficacies of polymyxin B and colistin against Francisella using an invertebrate waxworm model. Unfortunately, we found that polymyxin and colistin both killed the waxworms at concentrations predicted to be able to rescue the waxworms from Francisella infection, data in FIG. 6. This is not surprising given the known toxicity of these cyclic peptide antibiotics. Thus, we were not able to compare the ability of polymyxin and colistin to rescue the waxworm from Francisella infection.

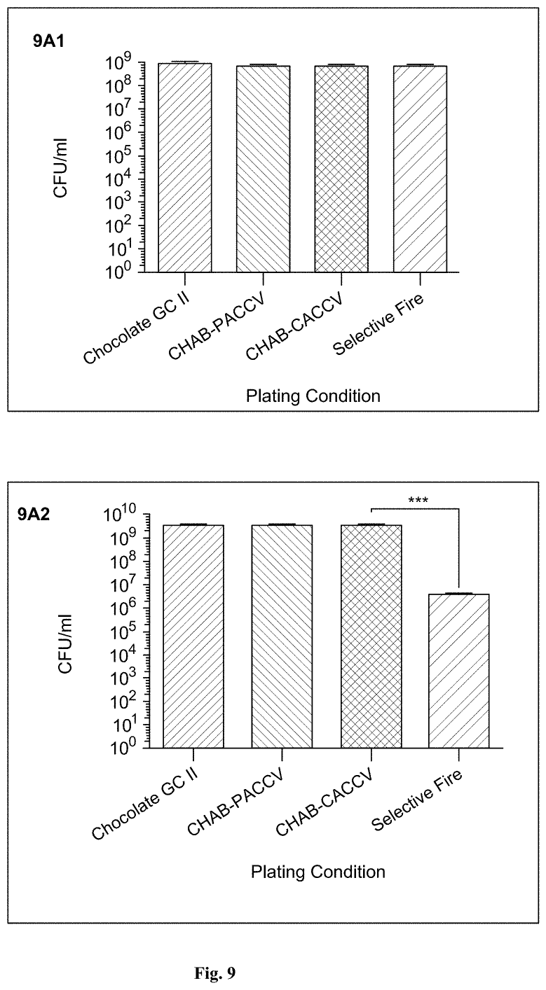

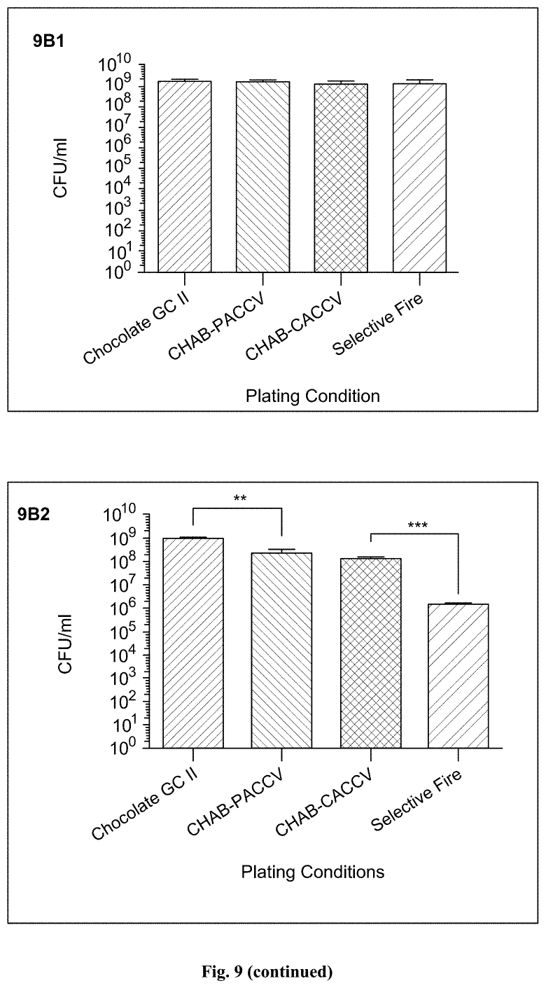

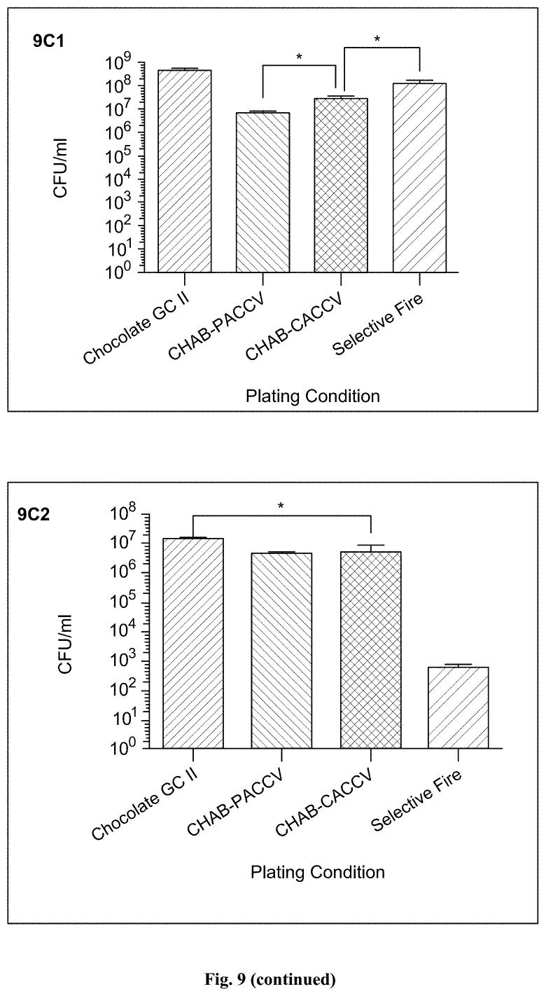

In an embodiment, we performed several tests to evaluate new formulations of Francisella selective media. We simultaneously evaluated replacing polymyxin B with colistin and using colistin with our novel FIRE base. One of the first tests performed was the ability of Francisella selective media ability to isolate Francisella from a complex mix of bacteria. Fast growing examples of potential environmental genera were added into a mixture with F. tularensis NIH B38, the slowest growing and most fastidious of the Francisella strains tested. A Candida strain did produce colonies on CHAB-CACCV, CHAB-PACCV and selective FIRE (FIG. 8A); however, NIH B38 was still able to be recovered when mixed with the Candida strain (FIG. 8B). Pseudomonas, Staphylococcus, Aspergillus, Escherichia, and Bacillus were all unable to grow on CHAB-CACCV and selective FIRE. All species tested were also streaked out onto GC II Chocolate agar to ensure their viability (FIG. 8C). F. novicida, LVS, F. philomiragia, F. tularensis NIH B38 and F. tularensis SchuS4 were also grown individually on CHAB-CACCV and selective FIRE medium (FIG. 8A). When grown on CHAB-CACCV, the Francisella species appear to leave a magenta halo around groups of colonies. This is evident in the first quadrant of a streak for isolation where the colonies are densely clustered. This is especially apparent in the fully virulent F. tularensis SchuS4.

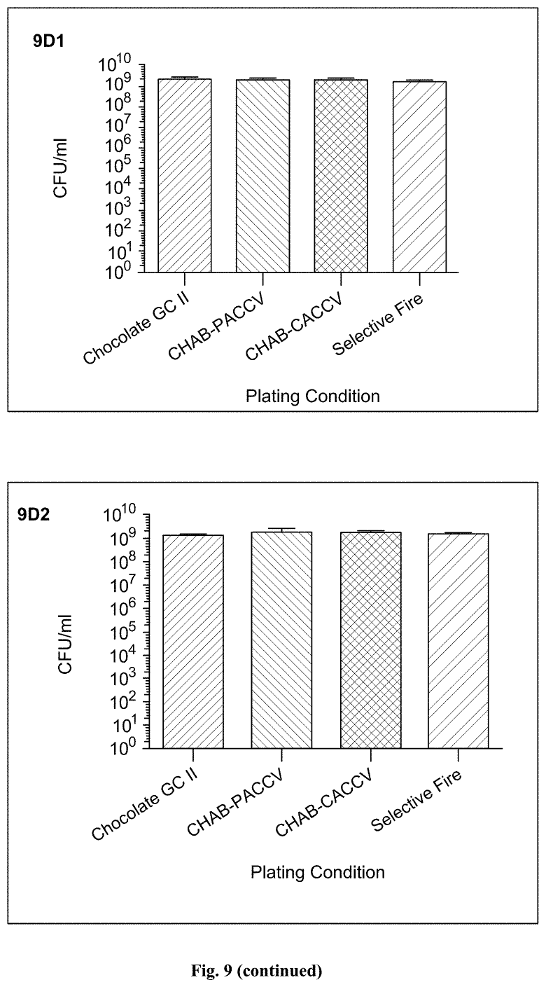

In an embodiment, we plated equal concentrations of bacteria onto each of GC II agar, CHAB-PACCV, CHAB-CACCV and Selective FIRE. It became apparent that the origin of the Francisella had a significant impact upon recovery. That is, the conditions of growth of the inoculating culture impacted the outcome of growth in the next step on different agars. We tested both bacteria that originated from a lawn grown on GC II chocolate agar (FIG. 9A) and bacteria that were originally grown in broth (FIG. 9B). This corresponds to anecdotal reports. When grown in TSBC, both F. tularensis LVS and F. novicida were recovered at equal rates by Chocolate GC II agar, CHAB-CACCV, CHAB-CACCV and Selective FIRE Whereas when grown as a lawn on Chocolate GC II agar F. novicida was recovered at equal rates on GC II agar, CHAB-PACCV and CHAB-CACCV, but was recovered at a significantly lower rate on selective FIRE plates. When the inoculation was grown as a lawn on Chocolate GC II agar LVS was recovered significantly more with GC II agar, than either CHAB-PACCV or CHAB-CACCV. Selective FIRE recovered significantly the fewer F. tularensis LVS colonies under these conditions than CHAB-PACCV and CHAB-CACCV. When grown in BMFC broth, F. tularensis NIH B38 was recovered the most with Chocolate GC II agar then Selective FIRE then CHAB-CACCV and finally CHAB-PACCV with all populations being statistically different.

In an embodiment, when F. tularensis NIH B38 was scraped off of Chocolate GC II agar this hierarchy changed with Chocolate GC II agar recovering the most followed by significantly fewer colonies being recovered by CHAB-PACCV and CHAB-CACCV (essentially the same for each) and finally, recovering many fewer logs of F. tularensis NIH B38 with selective FIRE. Finally, F. tularensis SchuS4 whether scraped from Chocolate GC II agar or grown in BMFC was able to recovered at equal rates by all four media tested here.

In an embodiment, Francisella (F.) tularensis is the causative agent of tularemia. A disease endemic to the US. It has also been weaponized in the past. Francisella is facultatively intracellular, which means it prefers to grow inside of other cells. Because of this growing Francisella in monoculture (by itself), especially the most dangerous strains, is difficult to do. Herein is a recipe and instructions for preparation of a growth medium (FIRE) that allows rapid growth of even the most fastidious strains of Francisella in monoculture in broth. While FIRE was designed for Francisella, it likely has applications with other hard to grow microbes.

In an embodiment, we set out to create a novel broth capable of growing both fully virulent SchuS4 and the type strain NIH B38. Achieving our goal, we created FIRE medium, which enables rapid growth across multiple Francisella strains. Performing growth curve analysis, we demonstrated that not only was this medium able to grow the fastidious NIH B38, it was also able to grow F. holarctiva LVS and F. novicida very well, outcompeting commonly used Francisella media such as TSB-C and BHI broth.

In an embodiment, FIRE also has a lower background absorbance than other media capable of growing NIH B38 such as BMFC which is quite dark (15), making it a more suitable medium for OD600 nm spectrophotometer based reads with lower theoretical standard deviations.

In an embodiment, it contains only one ingredient that is chemically undefined making it easier than other broths/agars (53) to optimize for various assays. Yet in total, there are only seven ingredients to be added to water making it significantly easier and less time consuming to make compared to chemically defined media that grow Francisella such as Chamberlains Defined Medium (54, 55). We were able to exclude blood from our medium. While blood is an important source of iron for Francisella (56), we elected to utilize ferric pyrophosphate as an alternative, which is cheaper than blood, does not require refrigeration, does not expire rapidly and does not need to be chocolatized into solution. This FIRE medium also results in the fastest growth of all the broths tested.

In an embodiment, FIRE has a low cost (Table 7), making it more affordable than some of the other alternative agars and broths (15, 28, 32, 34). All costs were calculated from the Sigma Aldrich website using the smallest available sized container to calculate cost/gram/ml except for the brain heart infusion which was calculated from US Biological Life Sciences website the Sheep blood which was calculated from the Quad Five website and the rabbit blood which was calculated from the Hemostat website. All costs are listed in US Dollars.

TABLE 7
Selective Media Cost Comparison.

| Medium | Ingredient | Ingredient Cost | Medium Cost |
|---|---|---|---|
| Remel | Casein Peptone | 8.13 | 59.74 |
| | Dextrose | 2.16 | |
| | Sodium Chloride | .39 | |
| | Yeast Extract | 1.09 | |
| | Beef Heart Infusion | .68 | |
| | L-Cysteine | .61 | |
| | Agar | 8.18 | |
| | Rabbit Blood | 33.00 | |
| | Penicillin | 1.75 | |
| | Polymyxin B | 3.75 | |
| CHAB-PACCV | Beef Heart Infusion | 3.40 | 71.66 |
| | Proteose Peptone | 3.26 | |
| | Dextrose | 2.16 | |
| | Sodium Chloride | .39 | |
| | L-Cysteine | .61 | |
| | Agar | 8.18 | |
| | Sheep Blood | 25.20 | |
| | Amphotericin | 1.19 | |
| | Cefepime | 18.36 | |
| | Cycloheximide | 6.30 | |
| | Vancomycin | .80 | |
| | Polymyxin B | 1.81 | |
| CHAB-CACCV | Beef Heart Infusion | 3.40 | 73.15 |
| | Proteose Peptone | 3.26 | |
| | Dextrose | 2.16 | |
| | Sodium Chloride | .39 | |
| | L-Cysteine | .61 | |
| | Agar | 8.18 | |
| | Sheep Blood | 25.20 | |
| | Amphotericin | 1.19 | |
| | Cefepime | 18.36 | |
| | Cycloheximide | 6.30 | |
| | Vancomycin | .80 | |
| | Colistin | 3.30 | |
| Selective FIRE | Meat Peptone | 6.59 | 49.74 |
| | Dextrose | 2.16 | |
| | Sodium Chloride | .78 | |
| | L-Cysteine | .07 | |
| | Agar | 8.18 | |
| | Ferric | .20 | |
| | Pyrophosphate | | |
| | Sodium Phosphate | 1.03 | |
| | Monobasic | | |
| | Monohydrate | | |
| | Sodium Phosphate | .78 | |
| | Dibasic Anhydrous | | |
| | Amphotericin | 1.19 | |
| | Cefepime | 18.36 | |
| | Cycloheximide | 6.30 | |
| | Vancomycin | .80 | |
| | Colistin | 3.30 | |

In an embodiment, the BSL2-Type A strain F. tularensis NIH B38 would not grow in autoclaved FIRE medium but would grow when the medium was prepared with filter sterilization instead. This effect was not seen in the other BSL2 Francisella strains tested. Given that this relationship may shed light on why BSL3 strains of Francisella are more fastidious than most BSL2 strains, we chose to investigate the phenomenon. Applicable concentrations of Maillard reaction products, which might be generating during autoclaving, were shown to be capable of inhibiting F. tularensis NIH B38, but not F. novicida or LVS. The exact product(s) that leads to this inhibition, the mechanism by which inhibition is induced and the role this might have in the life cycle of tularemia will be studied in future work. We believe this was not the main cause of growth inhibition in autoclaved FIRE as the Maillard products were created with only sugar and amino acid ingredients present, therefore the actual concentration of reactants available to participate in the Maillard reaction was likely higher in this solution than in the full autoclaved FIRE mixture as the sugar and amino acid ingredients likely interact with other ingredients as well. Also, the other ingredients being present may alter reaction kinetics by mechanisms other than flux diversion.

In an embodiment, to test for the possibility that some nutrients were sequestered or destroyed by autoclaving we complemented back in filter-sterilized FIRE medium at a 2× concentration. This restored growth to approximately 45%. This effect was not seen when simply diluting with deionized water suggesting that something in the filtered FIRE medium allowed growth. Either an ingredient added was needed for growth or an ingredient added countered some sort of toxic effect, either by reversing a reaction or sequestering it. This experiment was complicated by the fact that as the destruction of ingredients (which and what percentage) in the autoclave is unknown, we do not know if we perhaps added too much of an ingredient in which may have slowed growth. As all the combinations of solution that might be used in this experiment were intractable, we chose to complement this experiment with single elimination experiments.

In an embodiment, we eliminated each ingredient individually from autoclaved FIRE medium and attempted to culture NIH B38. Autoclaved FIRE medium without dextrose and autoclaved FIRE medium without sodium phosphate monobasic monohydrate allowed growth. Autoclaved FIRE medium without ferric pyrophosphate did not support growth. Further investigating this phenomenon, we tested autoclaved FIRE medium without ferric pyrophosphate and dextrose and found that this also supports growth. We also tested F. tularensis SchuS4 and obtained the similar results except the medium with sodium phosphate monobasic monohydrate did not support growth. The reason for this disparity is unknown. We believe in the autoclaved FIRE medium without dextrose and ferric pyrophosphate condition NIH B38 and SchuS4 are using trace amounts of iron from the meat peptone to grow or trace amounts of carryover from previous culturing conditions. According to the best available data we believe there is roughly 0.9 mg of iron added into 1 liter of FIRE medium from the meat peptone. We believe this is evidence that dextrose and iron interact in autoclaved FIRE medium to form something that is toxic by some mechanism. We then showed that the likely inhibiting compound can be made by autoclaving ferric-pyrophosphate and dextrose (dissolved in deionized water) in isolation.

In an embodiment, we identified several unique compounds formed in autoclaved FIRE including three antibiotic compounds and a detergent, lauryl sulfate were subsequently added back in and shown to be capable of significant growth inhibition.

In an embodiment, we then sought to develop our FIRE medium into a Francisella selective medium would exclude the growth of other organisms but would allow the growth of Francisella.

In an embodiment, polymyxins are cyclic peptide antibiotics produced as non-ribosomal peptides by bacteria. Polymyxins have fallen out of favor in clinical use due to the incidence of cytotoxicity, ototoxicity and nephrotoxicity (61). Recently, spurred on by the rise of multi-drug resistant bacterial strains, the polymyxins have become highly relevant and critical as drugs of last resort for multi-drug resistant infections. Three polymyxins have been tested against Francisella bacteria: polymyxin B (62), polymyxin E (colistin) (63), and polymyxin A (M) (mattocin) (64-66). Polymyxin B and colistin have clinical use for patients with multi-drug resistant gram-negative infections. Polymyxin B and colistin can both be applied topically, and polymyxin B is commonly found in “triple antibiotic” over-the-counter preparations. Colistin in the form of colistin sulfate is used in bacteriology experiments. Clinically, the hydrolysable precursor colistimethate sodium (CMS) is administered IV for systemic infections (65) and is hydrolyzed within the body to colistin, which is the active antimicrobial form.

In an embodiment, Francisella is highly resistant to cyclic peptide antibiotics such as polymyxin B (67). The MIC was reported to be approximately 800 ug/ml (68). The resistance of Francisella to polymyxin B is thought to be due to the special structure of the lipopolysaccharide (LPS) of Francisella (69). Thus, Francisella is considered to be inherently resistant to this class of cationic peptide antibiotics. It is reported that Francisella is also resistant to colistin (polymyxin E) in the form of colistin sulfate, but this has not been very well characterized, especially in the virulent strains (43). This polypeptide antibiotic is approved for clinical use and it has more history of recent clinical use than polymyxin B (70, 71).

In an embodiment, as Francisella strains are known to be highly resistant to polymyxins we sought to determine which of the two commonly available polymyxins was less inhibiting to Francisella. To this end, we tested the various resistances of three strains of Francisella to both colistin and polymyxin B using the CLSI MIC method. Testing NIH B38 using this method was not possible as the strain grows very poorly in liquid CA-MHC broth and agars (8). We used both an uncommon medium reported to be capable of growing the NIH B38 (15) and a new medium developed in our lab (FIRE medium) to allow us to more directly compare the resistances of strains using a broth dilution method (32, 45). All strains had greater or equal resistance to colistin than polymyxin B. We believe minor differences between our study and previous studies (68) are due to medium used and solvent used when preparing antibiotics. We then confirmed these relative resistances with a disk inhibition method while simultaneously showing the generation of Polymyxin B resistant F. novicida mutants. As the resistance to colistin was always higher or equal to that of polymyxin B and the killing kinetics showed little difference, we chose to replace polymyxin B with colistin in our selective medium.

In an embodiment, to clearly demonstrate the utility of both our new Francisella selective agar we chose to test our ability to isolate the slow growing fastidious NIH B38 Francisella strain from a complex mix of microorganisms, as it can easily be overgrown by a myriad of other species in permissive conditions. We were able to isolate NIH B38 from a complex mixture of common environmental microbial contaminants and exclude all contaminants completely except for a strain of Candida, which grew on both CHAB-CACCV and Selective FIRE medium. The fully virulent Francisella tularensis SchuS4 and BSL2 model organisms F. novicida and LVS could also be cultured on these media.

In an embodiment, in order to determine the relative efficiency of recovery, we decided to test the quantitative recovery of each plate side by side. It became clear that the origin of the Francisella culture spread on the plate had a strong effect on subsequent recovery. Francisella grown in the broths were recovered significantly better on selective FIRE than compared to chocolate agar-based selective plates. The opposite effect with a larger magnitude was seen when Francisella was first cultured on non-selective GC II chocolate type plates. This trend remained true for all strains tested but at different orders of magnitude.

In an embodiment, the previous culturing method of Francisella affects their ability to grow on subsequent media. The reason for the “chocolate-FIRE” effect is not immediately clear. The reason for the strain disparities is also not immediately clear. We hypothesize that the “chocolate-FIRE” effect may have to do with a metabolic shift when going from one medium to the next as Francisella is known to undergo significant proteomic change when grown in different media (72), and the original medium Francisella is cultured in is known to influence subsequent downstream in vivo tests (73). Also, the aging of organisms seems to have different effects on the infectivity of Francisella depending upon route of infection (74). This theory may also be expanded to encompass lipid or carbohydrate profile changes. Other “memory effects” have been seen in regards to antibiotic persister properties in other bacteria (75). Subsequent studies will be necessary to investigate these and additional possible explanations for the chocolate-FIRE effect.

In an embodiment, given the utilities of our new medium and its use in selective media we have named it FIRE (Francisella isolation, recovery, and enrichment) medium. We have shown that this media enables the growth of the Francisella Type A type strain (NIH B38) in liquid broth at BSL2. We have shown that selective FIRE medium can recover Francisella at similar rates to other Francisella selective media. This novel medium is significantly less expensive to make than other Francisella selective media. As colistin resistance is higher or equal to that of polymyxin B in all strains tested, we hypothesize this pattern may be applicable to any new Francisella strains that may be yet discovered. We recommend the use of FIRE medium for the selective culturing of Francisella based upon its economic feasibility, its robust recovery and its potential to not exclude novel Francisella strains

In an embodiment, Francisella tularensis is the causative agent of tularemia. Tularemia is of interest because it is endemic to the USA and it has been developed into a biological weapon by many nations. Therefore, there is a robust Francisella research community pursuing the non-exclusive infectious disease and biodefense research paths. When studying a microorganism traditionally the first goal Is learning how to culture (grow) the organism in monoculture (by itself).

In an embodiment, Francisella is facultatively intracellular, which means it prefers to live inside of other cells. This makes culturing Francisella by itself challenging. As rule of thumb the more infectious and dangerous the strain of Francisella (and therefore the strains most relevant to study) the harder it is to culture.

In an embodiment, the methods for culturing these particularly fastidious Francisella strains are slow inconvenient and expensive. We have invented a new medium capable of the rapid growth of even the most fastidious Francisella strains quickly. This medium is called Francisella Isolation Recovery and Enrichment (FIRE) based on its utilities when growing Francisella. FIRE is cost effective, light in color, relatively easy to make, does not need to be refrigerated, and can be a broth.

In an embodiment, recipe is for making one liter of selective fire agar (Table 1). This is the most elaborate form alternative embodiments will be explained afterward. Into 500 ml of deionized water add all of the ingredients listed. Wait all ingredients except ferric pyrophosphate are completely dissolved. Heat may be applied if one is in a hurry. The amount of ferric pyrophosphate in the recipe exceeds its solubility so some will be filtered out in the next step. Given the cost of ferric pyrophosphate (also known as iron phosphate) and the difficulty of measuring out miniscule masses the perfect amount is unknown. This is now a 2× solution of fire. Prolonged storage of this will result in crashing and thus should be avoided. Filter sterilize the 2× solution into a sterile storage container. In a one-liter bottle add 15 g of agar in 500 ml of deionized water. Dissolve/sterilize (if so desired) the agar into the water. Next add the 2× solution to the agar when the agar is 55° C. Now add the antibiotics in to the right concentrations. Proceed to pour the agar into the desired vessel(s). This selective medium would be used to isolate Francisella and exclude other organisms that could not grow in the presence of the antibiotics.

In an embodiment, the antibiotic mixture is prepared based upon previous studies (1,2). Remove the agar and make a 1× selective broth. Remove the antibiotics and make a non-selective plate. Remove both and make a 1× non selective broth. Reduce the agar percentage to make less solid plates for motility studies or other such endeavors. Change out the agar with another solidifying agent such as Gel-Gro. The 2× scheme could also be reworked to other values for example one could make a 1.5×FIRE solution in 666 ml and add that into 333 ml of molten agar solution.

In an embodiment, there are other general adjustments one could make to the FIRE which would allow its practice although with slightly altered characteristics. Water could be added to powder to get a final volume of 1 liter as opposed to the water being the first ingredient which results in a volume over 1 liter and therefore a more dilute medium. Also the buffering system (sodium phosphates could be switched) or the peptone ingredient (meat peptone) could be swapped. The carbon source (dextrose) could also be swapped. Also the whole mixture could be autoclaved although this will reduce its utility in culturing the most fastidious Francisella strains. Alternatively, all ingredients could be autoclaved except for ferric pyrophosphate or dextrose or both and then those filter sterilized ingredients could be added back in afterwards. The mass of any ingredients added could be moved a bit up or down. Many of the ingredients could be excluded and the medium would still be functional albeit often at a reduced capacity depending upon the strain tested against.

As a rule of thumb all liquid broths can be converted into solid plates, but not all solid plates can be converted into broths. Broths are often desirable as they require less work to create and handle, they are also by their nature cheaper than agars.

Turbidity is a common measurement of cell growth and darker media make doing so more challenging. In an embodiment, medium is easier and cheaper to make than its competitors.

Creating media is generally assigned to less senior personnel as it is usually routine work. Unfortunately, with Francisella many of the media require more seasoned researchers to devote their time to this pursuit. In an embodiment, FIRE considerably lowers the skill bar for culturing the most fastidious Francisella strains.

In an embodiment, this medium does not require refrigeration.

In an embodiment, this media is to grow Francisella for whatever purpose.

In an embodiment, to the best of our knowledge FIRE results in the shortest time until measurable growth in Francisella when compared to all known competitors. The present application is about a new growth medium, FIRE, for the growth of Francisella tularensis. This media does not use blood products, is easy to make, stores well, and is significantly less expensive than the current options. The use of the BSL-2 type strain F. tularensis NIH B38 for tularemia studies has been enabled and further demonstrated the benefit of using NIH B38 by discovering a novel inhibitory compound that inhibits the growth of F. tularensis tularensis SchuS4.

In an embodiment, FIRE has been shown to be amazing for Francisella work, but it likely has applications in growing other fastidious genera such as Mycobacteria, Yersinia, Coxiella, Rickettsia, Salmonella, Brucella, Listeria, Shigella, Chlaymdia, Shigella, Legionella, Helicobacter, Neisseria. While it is unlikely FIRE will support robust growth in all of these genera, it is incredibly likely it will work well in some of them. The medium was designed to simulate being inside of a mammalian host. When searching for other bacteria the medium might be optimal for, I would include phrases like “fastidious” “facultative intracellular” and “obligate intracellular”.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### Example 1

Bacterial strains and growth conditions: The following strains of bacteria were used in this study: F. tularensis subsp. novicida U112; F. tularensis subsp. holarctica CDC LVS NR-646; F. tularensis tularensis NIH B38 (NR50, derived from ATCC 6223, FSC230, BEIResources.org); F. tularensis tularensis SchuS4 (NR10492, BEIResources.org). Francisella novicida and LVS were propagated from glycerol stocks in cation adjusted Mueller-Hinton broth (CAMHB) (Becton Dickinson, Cockeysville, Md., USA) for the MIC assays. Francisella tularensis NIHB-38 was propagated for growth curves from glycerol stocks in a BMFC type medium (15) which enables the growth of this strain in liquid medium for MIC assays, which is reported to be otherwise difficult (15, 16). This medium was made by adding 10 ml of autoclaved 2.5% (w/v) ferric pyrophosphate (Sigma) solution and 2.5 ml of a 50 mg/ml filtered stock solution of L-cysteine (Alfa Aesar, Ward Hill, Mass., USA) into a previously autoclaved solution of 987.5 ml deionized water with 37 g of brain heart infusion (Becton Dickinson Franklin Lakes, N.J., USA) and 22 g of CAMHB. Francisella tularensis SchuS4 NR10492 was propagated from frozen single-use glycerol stocks onto Chocolate II agar (GC II agar with Hemoglobin and IsoVitalex. 221267, Becton Dickinson) (unless otherwise stated) into either pH 6.8 Brain Heart Infusion broth (Teknova, Hollister, Calif., USA) or onto subsequent GC II Chocolate agar.

Other bacteria for competition assays: Pseudomonas aeruginosa PA01, Bacillus subtilis 168, Candida albicans Robin Berkhout ATCC MYA-2876, Staphylococcus aureus 25923, Escherichia coli K12, and F. philomiragia ATCC 25017 were all obtained from ATCC and grown in 5 ml of Tryptic soy broth with 0.1% cysteine (TSBC) at 37° C. CO2. Aspergillus niger Ward 470177-276 was obtained from Carolina Biosciences and was grown at 28° C. in 5 ml of TSBC for 4 days.

FIRE Broth and Agar Compositions: FIRE medium is made as follows.

Given the results of this study we hypothesize as an alternative to filter sterilizing, FIRE may also be made by autoclaving all ingredients and adding in filter sterilized glucose afterwards.

When FIRE Agar Plates were made a solution of 2× Fire was filter sterilized and was then diluted 1:2 into autoclaved agar.

Bacterial Growth Curves: F. novicida U112 and F. holarctica LVS were cultured in TSBC whereas NIH B38 was cultured in BMFC to generate the inoculum for the 96 well plates. A concentration of cells equaling 5*105 was used for each inoculum. Growth curves were conducted in a Biotek 800 TS Absorbance Reader which was set to 37° C. Samples were incubated kinetically for 72 hours in normal atmosphere with absorbance being measured at OD600 every 15 minutes.

Destroyed Nutrient Testing: In a 96 well plate format growth of NIH B38 was tested for growth in filter-sterilized FIRE, autoclaved FIRE, half strength autoclaved FIRE (diluted with deionized water) and half autoclaved FIRE half 2× filter-sterilized FIRE. Growth was measured as absorbance at OD600 nm with a spectrophotometer after 2 days of static incubation at 37° C. with 5% CO2.

Autoclave Product Inhibition/Limitation Testing: Caramelization products were made by autoclaving 2 grams of glucose in 100 ml of deionized water by adding 10.5 grams of meat peptone, 10 milligrams of L-Cysteine Hydrochloride and 2 grams of glucose to 100 ml of deionized water. These solutions were autoclaved for 20 minutes at 121 degrees Celsius at a pressure of 15 psi. Solutions were diluted one to two in filter-sterilized FIRE. One to two dilutions were performed serially across the 96 well plates. Growth was ascertained visually and by spectrometer daily for period of 2 weeks. Filter-sterilized FIRE was used as a negative control and inoculated filter-sterilized FIRE was used as a positive control. A Maillard plate sans organisms was used to control for browning. For pH, concentrated hydrochloric acid was utilized to acidify the medium for testing. After serial dilution, pH of each sample was determined empirically using an AR25 Accumet pH meter. 100 μl of deionized water added into previously inoculated 100 μl of FIRE was also included as a positive control. Growth for these experiments was measured as absorbance at OD600 nm with a spectrophotometer after 2 days of static incubation at 37° C. with 5% CO2.

Single/Double Elimination Experiments: All of the single and double elimination FIRE solutions were made in volumes of 100 ml of deionized water. In the event that precipitate occurred it was allowed to settle, and liquid was removed from the top to conduct testing. Bacterial strains for this test were grown in 5 ml of BMFC. Inocula of 5×10\hat{ }5 CFU/ml were added into a total of 200 μl of each solution in a 96 well plate. The plates were incubated statically at 37° C. with CO₂ for two days. Growth was then measured as absorbance at OD600 with a spectrophotometer.

Quadrupole Time of Flight Mass Spectrometry: Samples were prepared and spectra were measured.

Minimum Inhibitory Concentration (MIC) Assay: The Minimum Inhibitory Concentration is defined as the lowest concentration of antibiotic tested that prohibited any Francisella bacterial growth as measured by OD₆₀₀ nm and the assays were performed following the Clinical & Laboratory Standards Institute (CLSI) guidelines (31). MIC testing was conducted in flat bottom clear 96 well plates (Falcon 353072, Becton Dickinson). Each antibiotic was stored at 4° C. as a 10 mg/ml stock solution in sterile deionized water. The relevant antibiotic was diluted serially two-fold across the plate starting at a concentration of 1024 μg/ml. Colistin sulfate was obtained from Research Products International (Mt Prospect, Ill., USA) and polymyxin B was obtained from MP Biomedicals LLC (Solon, Ohio, USA). The MICs were performed with CAMHB with 2% IsoVitalex. This solution was set to a pH of 7.1 (32) except for NIH B38 where MICs were conducted in FIRE (Francisella isolation, recovery, enrichment) and BMFC identical MIC values). Inoculation of 5×10⁵ CFU/ml was used in these assays. Cell density was read in a McFarland Densitometer. Inncola were adjusted to be equivalent to 5*10⁵ CFU/ml which corresponds to an estimated McFarland value of 0.0003 for F. novicida in CAMHB, 0.0004 for LVS in CAMHB and 0.0006 for NIH B38 in BMFC/FIRE. The total volume of each experimental well was 200 μl. All plates were incubated at 37° C. at 5% CO₂. The OD₆₀₀ of all plates was measured at 48 hours.

Generation of Francisella Lawns: F. novicida and LVS were grown for 1 day whereas NIH B38 was grown for two days in FIRE. To generate the lawn, 5 ml of culture was spread over a Chocolate GC II plate. This was allowed to sit in the biosafety cabinet with the lid closed for 40 minutes. Subsequently, all liquid was removed and plates were incubated at 37° with CO₂ incubated for two days yielding robust bacterial lawns.

Generation of Spontaneous mutants on agar: Similar to the method we previously used to identify Francisella resistance to fosmidomycin (10, 33), 6 mm paper disks were soaked in a 10 mg/ml solution of either Polymyxin B or Colistin dissolved in water. Following the removal of excess liquid from lawn-making procedure, these disks were placed in the center of the plates. Plates were subsequently incubated at 37° C. CO₂ for 2-4 days, and were observed for the growth of any colonies inside the zone of inhibition, which might suggest resistance mutations.

Galleria mellonella infection and treatment: Galleria mellonella (waxworm) larvae (Vander Horst Wholesale, St Mary's OH, USA) were used to assess the toxicity of colistin or polymyxin B which may have rescued the host from Francisella infection. The caterpillars were used in their larval stage. The caterpillars were stored at room temperature in the dark. The populations utilized were injection free, phosphate buffered saline (PBS) injection, and two-fold dilutions of polymyxin B/colistin starting at 1024 μg/ml and ending at 128 μg/ml which were diluted in cell culture PBS. The total volume of each injection was 10 μl which was given into the left proleg of each animal using a 27.5-gauge needle. Caterpillars were then incubated at 37° C. for five days with corpses counted daily. The mean time until mortality was determined. This data was graphed using GraphPad Prism.

Characterization of CHAB-CACCV and FIRE

i. Competitive Isolation

Pseudomonas aeruginosa PA01, Bacillus subtilis 168, Candida albicans Robin Berkhout ATCC MYA-2876, Staphylococcus aureus 25923, Escherichia coli K12, F. novicida, LVS, and F. philomiragia ATCC 25017 were all grown in 5 ml of Tryptic soy broth with 0.1% cysteine (TSBC) at 37° C. CO2. NIH B38 was grown in 5 ml of FIRE at 37° C. CO₂ Aspergillus niger Ward 470177-276 was grown at 28° C. in 5 ml of TSBC for 4 days. 100 μl of each culture was pelleted and the supernatant was removed. The pellets were then resuspended in PBS. A streak for isolation was performed using each of these suspensions on permissive agar and on selective agar. Following this, suspensions were mixed as indicated and plated upon our selective agars.

ii. Comparative Recovery Yield from Lawns: F. novicida, LVS, NIH B38 or F. tularensis SchuS4 were scraped from a lawn into 2 ml of BHI (Teknova) until an increase of 0.5 McFarland units over background was reached. These solutions were serially diluted with BHI medium and plated in triplicate at three separate dilutions using GC II Chocolate agar, CHAB-PACCV, CHAB-CACCV and Selective FIRE. Plates were incubated statically at 37° C. with 5% CO2. Colonies were counted after 2-3 days of incubation.
iii. Comparative Recovery Yield from Liquid Cultures: F. novicida and LVS were grown in TSBC and NIH B38 and F. tularensis SchuS4 was grown in BMFC. These were then serially diluted in their respective broths and immediately plated with dilutions in triplicate upon GC II Chocolate agar, CHAB-PACCV, CHAB-CACCV and Selective FIRE. Plates were incubated statically at 37° C. with 5% CO2. Colonies were counted after 2-3 days of incubation.

Biosafety: All the Francisella strains were handled at the required biological safety level, either BSL2 or BSL3, as appropriate. All experimental procedures were approved by the respective Institutional Biosafety Committees.

Statistical analysis: All microbiology experiments were repeated at least three independent times with multiple technical replicates as indicated. All statistically different populations were asserted by students t test (paired tails, homoskedasticity) using a confidence interval of 95%.

### REFERENCES

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof
Chamberlain, R. E. (1965). Evaluation of live tularemia vaccine prepared in a chemically defined medium. Applied microbiology, 13(2), 232-235.
Franz D R, Jahrling P B, Friedlander A M, McClain D J, Hoover D L, Bryne W R, Pavlin J A, Christopher G W, and Eltzen E M, Jr. (1997). Clinical recognition and management of patients exposed to biological warfare agents. JAMA 278:399-411.
Alharby, A. M. (2014). Isolation and characterization of a novel bacteriophage, ASC10, that lyses Francisella tularensis (Doctoral dissertation, Colorado State University. Libraries).
Morris, B. J., Buse, H. Y., Adcock, N. J., & Rice, E. W. (2017). A novel broth medium for enhanced growth of Francisella tularensis. Letters in applied microbiology, 64(6), 394-400.
Mc Gann, P., Rozak, D. A., Nikolich, M. P., Bowden, R. A., Lindler, L. E., Wolcott, M. J., & Lathigra, R. (2010). A novel brain heart infusion broth supports the study of common Francisella tularensis serotypes. Journal of microbiological methods, 80(2), 164-171.
https://legacy_bd.com/ds/technicalCenter/inserts/L007361(09).pdf.
Petersen, J. M., Carlson, J., Yockey, B., Pillai, S., Kuske, C., Garbalena, G., . . . & Chalcraft, L. (2009). Direct isolation of Francisella spp. from environmental samples. Letters in applied microbiology, 48(6), 663-667.
Petersen, J. M., Schriefer, M. E., Gage, K. L., Montenieri, J. A., Carter, L. G., Stanley, M., & Chu, M. C. (2004). Methods for enhanced culture recovery of Francisella tularensis. Applied and environmental microbiology, 70(6), 3733-3735.

1. A composition comprising:
a growth medium comprising a carbon source, a nitrogen source, a sulphur containing amino acid and a Fe containing salt comprising Ferric pyrophosphate;
wherein the growth medium has no blood; wherein the growth medium is configured to allow growth of a living microorganism.

2. The growth medium of claim 1, wherein the sulphur containing amino acid comprises cysteine.

3. The growth medium of claim 1, wherein the growth medium is configured to growth of Francisella.

4. The growth medium of claim 1 wherein the carbon source comprises at least one of Lactose, Sucrose, Dextrose.

5. The growth medium of claim 1, wherein the nitrogen source comprises at least one of peptone, Beef extract, Casein Hydrolysate, Yeast Extract.

6-7. (canceled)

8. The growth medium of claim 1, further comprises a salt to regulate an osmotic stress.

9. (canceled)

10. The growth medium of claim 1, wherein the growth medium is configured to allow growth of a BSL-2 strain of Francisella and a BSL-3 strain of Francisella.

11. The growth medium of claim 1, further comprises thiamine.

12. The growth medium of claim 1, further comprises spermine.

13. The growth medium of claim 1, wherein the growth medium is configured to convert into a selective growth media.

14. The growth medium of claim 13, wherein the selective growth medium comprises an antibiotic.

15-19. (canceled)

20. The growth medium of claim 14, wherein the antibiotic comprises at least one of polymyxin, colistin, Amphotericin, Cefepime, Cycloheximide, Vancomycin.

21-22. (canceled)

23. The growth medium of claim 1, wherein the Ferric pyrophosphate is more than zero and less than 1 g/l.

24. The growth medium of claim 1, wherein the growth medium has pH about 5 to about 7.

25. The growth medium of claim 1, wherein the growth medium does not require refrigeration for storage.

26. A method comprising:
mixing a nitrogen source, cysteine, a salt to regulate to osmotic water and a buffer to form a mixture;
adding water to mixture and autoclaving a dissolved mixture in the water; and
adding ferric salt and a carbon source to form a growth medium;
wherein the growth medium has no blood and is configured to support growth of Francisella.

27. The method of claim 26, wherein the growth media is capable to be converted into a selective growth media by addition of an antibiotic.

28-30. (canceled)

31. The method of claim 26, wherein the growth medium is configured to allow growth of a BSL-2 strain of Francisella and a BSL-3 strain of Francisella.

32-33. (canceled)

34. The method of claim 26, wherein the growth medium has a pH of about 5 to about 7.

35. (canceled)

36. The method of claim 26, wherein ferric salt comprises ferric pyrophosphate.
